(19) United States
(12) Patent Application Publication
(10) Pub. No.: US 2025/0001064 A1
(43) Pub. Date: Jan. 2, 2025

(54) Quantifying Blood Loss By Recirculating Waste Fluid With A Medical Waste Collection System

(71) Applicant: Stryker Corporation, Kalamazoo, MI (US)

(72) Inventors: Stephen Faul, Cork (IE); Gerard W. Nunan, Bishopstown, Cork (IE); Michael Zollinger, Chelsea, MI (US); Brian MacLachlan, Norton Shores, MI (US); Brian James VanDerWoude, Portage, MI (US); Leif Norland, St. Joseph, MI (US); James T. Carroll, Midleton, Cork (IE)

(73) Assignee: Stryker Corporation, Kalamazoo, MI (US)

(21) Appl. No.: 18/704,578

(22) PCT Filed: Oct. 25, 2022
(86) PCT No.: PCT/US2022/047685

§ 371 Date: Apr. 25, 2024

Related U.S. Application Data
(60) Provisional application No. 63/271,763, filed on Oct. 26, 2021.

(51) Int. Cl. A61M 1/00

(52) U.S. Cl. CPC A61M 1/73; A61M 1/72; A61M 2205/121; A61M 2205/3306; A61M 2205/60

(57) ABSTRACT

Quantifying blood loss with a medical waste collection system. A removable cartridge forms a fluid path with a cartridge receiver, and defines a detection window that is optically clear. The cartridge may include a spine extending from a head and including opposing sides that are optically clear. A sensor module includes emitters and sensors configured to detect an optical characteristic of the waste fluid. A controller may determine the volume of blood loss based on blood concentration and volume of collected waste fluid. A sensor housing may be removably coupled to the spine of the cartridge. The cartridge may define a fluid reservoir, and include an actuator for drawing the waste fluid into the fluid reservoir. A tank may define the fluid reservoir. The waste fluid may be recirculated through a removable manifold. Methods of arranging and operating the medical waste collection system are also disclosed.

### PRIORITY CLAIM

This application claims priority to and all the benefits of U.S. Provisional Application No. 63/271,763, filed Oct. 26, 2021, the entire contents of which are hereby incorporated by reference.

### BACKGROUND

A byproduct of some surgical procedures is the generation of liquid, semisolid, and/or solid waste material. The liquid waste material may include bodily fluids and irrigating solution(s) at the surgical site, and the solid and semisolid waste material may include bits of tissue and pieces of surgical material(s). The medical waste, regardless of its phase, is preferably collected so it neither fouls the surgical site nor becomes a biohazard in the medical suite in which the procedure is being performed.

The medical waste may be removed from the surgical site through a suction tube under the influence of a vacuum provided by a medical waste collection system. One exemplary medical waste collection system is sold under the tradename Neptune by Stryker Corporation (Kalamazoo, Mich.). A manifold may be provided that facilitates interfacing the suction tube with the medical waste collection system. The manifold may be disposable.

The collected liquid waste material may include blood, and the blood may be in a suction path with other bodily fluids such as interstitial fluid, mucus, bile, and the like. Determining blood loss during surgery may be used to monitor patient health. Excessive blood loss may be indicative of surgical complications, and determining blood loss facilitates assessing transfusion requirements. Of particular interest is childbirth, wherein obstetric hemorrhage is a major cause of maternal morbidity. Earlier detection of obstetric hemorrhage may significantly reduce the maternal morbidity rate. There has been a growing impetus among clinicians and governing bodies to increase the usage and accuracy of blood loss quantification methods and tools, especially for vaginal and Caesarean deliveries where postpartum hemorrhage is of vital concern.

It is known to estimate blood loss during surgery by visually evaluating of absorbent articles (e.g., sponges, surgical gowns, bedding, or drapes), measuring the absorbent articles using a scale, and/or observing graduated collection vessels in the operating theatre. The aforementioned methods may provide suboptimal accuracy and appreciable delay from the blood loss from the patient and the determination of the blood loss.

Therefore, there is a need in the art for improved systems, devices, and methods for performing quantitative blood loss (QBL) analysis in an accurate, instantaneous, and repeatable manner.

### SUMMARY

The present disclosure is directed to performing QBL analysis using a medical waste collection system. The medical waste collection system includes a waste container, and a vacuum pump configured to draw the waste fluid through a suction line and into the waste volume. The medical waste collection system recirculates the waste fluid from the waste volume to be analyzed for the QBL analysis. At least one manifold receiver is supported on the chassis and defines a manifold opening sized to removably receive a manifold. The vacuum generated by the vacuum source is drawn on the suction tube, and the waste fluid at the surgical site is drawn through the manifold to be collected in the waste container. The vacuum source or another pump may circulate the waste fluid from the waste volume through a fluid path to be recollected within the waste volume. A sensor module is configured to detect an optical characteristic of the waste fluid being circulated in the fluid path.

The waste collection system may include a cartridge receiver configured to removably receive a cartridge to facilitate the QBL analysis. The cartridge receiver may be separate from the manifold receiver. The cartridge receiver may include a cartridge opening defined by a sensor housing or another housing. A sensor module is coupled to the sensor housing and includes at least one emitter and at least one sensor. The emitters are configured to emit energy, and the sensors are configured to detect the emitted energy. The emitters may be light emitting diodes (LEDs) and the sensors may be optical detectors. The sensors detect the emitted light, and more particularly the light after being transmitted or scattered through the waste fluid. A first emitter may be an infrared LED, and the second emitter may be a visible-light LED, such as a green LED. A first sensor may detect transmitted light and scattered light from the first emitter, and a second sensor may detect transmitted light and scattered light from the second emitter. The four measurements—two of the transmitted light and two of the scattered light—are values provided to a controller (or another processor) to perform the QBL analysis. An alternative arrangement includes two emitters and four sensors with each of the four sensors configured to detect one of the four measurements. There may be more or less than two emitters with the additional emitters configured to detect optical characteristics of non-blood fluids, such as amniotic fluid, bile, or the like. The emitters and sensors may be arranged adjacent to or opposite the fluid path in any suitable configuration. The detected intensity of the transmitted and/or the scattered light may be indicative of the transmissivity, opacity, and/or other physical property of the waste fluid. The cartridge may define a detection window forming a portion of the fluid path such that, with the cartridge removably inserted into the cartridge receiver, the detection window of the cartridge is positioned between the emitters and the sensors. A controller is configured to determine a blood concentration of the waste fluid based on the measurements.

A fluid measurement system is disposed within the waste container and in electronic communication with the controller. The fluid measurement system is configured to generate a waste level signal representative of a fluid level of the waste fluid collected within the waste container, and transmit the waste level signal to the controller. The controller determines a volume of the waste fluid based on the waste level signal. The controller may then further determine the volume of blood loss based on the blood concentration of the waste fluid and the volume of the collected waste fluid.

The user may select on a control panel operating parameters of the medical waste collection system, such as the vacuum level drawn on the waste container. The controller is configured to control the vacuum source and/or the vacuum regulator to operate the medical waste collection system based on the operating parameters. The control panel may further display the operating parameters and other information, such as the volume of blood loss as determined by the controller. A front of the chassis may define a window to permit a user to view the contents within the waste volume of the waste container.

The fluid path may define an inlet and an outlet each in fluid communication with the waste volume. A first conduit may be coupled to an exterior of the waste container to define the inlet, and a second conduit may be coupled to the exterior of the waste container to define the outlet. The pump and valves may be arranged in fluid communication with the waste volume and configured to circulate the waste fluid from the waste volume through the fluid path to be recollected within the waste volume. The pump may be coupled to the first or second conduits to define a portion of the fluid path. The pump may provide a positive or negative pressure to circulate the waste fluid from the waste volume through the fluid path. The sensor housing may also be coupled to the first or second conduits and define a portion of the fluid path.

The medical waste collection system may include an agitator disposed within the waste container. The agitator may be an impeller coupled to a motor for mixing the waste fluid. The agitator facilitates homogenization of the waste fluid within the waste volume and the fluid path. The agitator may be in communication with the controller, and the controller 36 may selectively operate the agitator based on any number of operating parameters. The controller may toggle off and on the agitator for fixed or varied intervals. In instances where the instantaneous blood concentration values deviate from one another greater than a predetermined threshold for a certain period of time, the controller may initiate operation or speed up operation of the agitator. Additionally or alternatively, the controller may operate or speed up the agitator during any periods in which the pump is being operated indicative of a QBL operating mode. The user may also provide an input to the control panel to selectively operate the agitator as needed. Circulating the waste fluid through the fluid path may itself facilitate homogenization of the waste fluid within the waste volume. The redepositing of the waste fluid within the waste container, perhaps from above the fluid level, may provide a further agitative effect.

The inlet of the flow path may be located below the outlet such that the pump is configured to pump the waste fluid through the fluid path against gravity. The waste container may be prefilled with the waste fluid or another fluid, such as water, to a fluid level above the inlet of the fluid path prior to circulating with the pump the waste fluid through the fluid path or activating the sensor module. The prefilled fluid level may be coordinated by the fluid measuring system and the controller. The waste fluid may also be diluted with the waste fluid or another fluid, such as water, such that the blood concentration is below a predetermined blood concentration.

The cartridge is removably inserted into the cartridge receiver and includes a cartridge housing sized to be inserted through the cartridge opening. The cartridge housing may be at least partially disposed within the sensor housing. The cartridge housing defines a channel configured to form or complete the fluid path with the cartridge removably coupled to the cartridge receiver. The cartridge housing may include shoulders each defining a bore. The channel may be coupled to the shoulders to define the passageway in fluid communication with the bores. The channel may itself define the detection window to be positioned between the emitters and the sensors when the cartridge housing is at least partially disposed within the void. The channel may be a tube or a member with another suitable cross section. The cartridge housing may define the detection window through cutouts or other geometries formed within the cartridge housing. The cartridge may include a handle with the shoulders extending from the handle. The handle may be positioned exterior to casing of the chassis to facilitate the cartridge being inserted into and removed from the cartridge receiver. Further, the cartridge may include retention features configured to releasably engage complementary retention features of the cartridge receiver. The retention features may form an interference fit or friction fit, or a movable structure configured to be actuated between engaged and disengaged positions. The actuation between the engaged and disengaged positions may be provided through a mechanical input on the chassis, or an input on the control panel in which the controller mechanically or electronically moves the structure to the disengaged position to permit removal of the cartridge.

The cartridge housing may include a trunk, and a head removably or fixedly coupled to the head to define a cartridge volume. The trunk may include a body portion, a first leg extending from the body portion, and a second leg extending from the body portion. A void may be defined between the first leg and the second leg. Each of the first leg and the second leg may extend proximally from the body portion to define the void therebetween.

The cartridge housing may include at least one of an arm, a lock element, a spine, and a catch. The arm and/or the spine may extend from the body portion or the first leg. The lock element may extend from the body portion, and the catch may be disposed on the second leg. The first leg may include a rim defining an outlet opening and/or a recirculation opening, and the second leg may define the other of the outlet opening and the recirculation opening. A first seal may be disposed within the outlet opening, and a second seal may be disposed within the recirculation opening. The first seal and the second seal may be similar or different in size, shape, material selection. One of the outlet opening and the recirculation opening may be positioned below the other with the cartridge is oriented for insertion into the cartridge opening of the cartridge receiver. The arm, the lock element, the spine, and the catch may be positioned distal to the outlet opening or the recirculation opening defined in the second leg.

The cartridge housing may define the detection window that is optically clear. The head may include a ridge that defines the detection window. The head may further define a recess that is circular with the ridge extending through the recess. The ridge includes opposing outer sides that are at least partially transparent. The cartridge may include a grip coupled to the head, the trunk, or another suitable structure of the cartridge.

The pump is operated to establish the fluid path from the waste volume and through the recirculation opening towards and through the detection window, and towards and the through the outlet opening to be recollected in the waste container. The pump may be arranged to draw a vacuum on the outlet opening to draw the waste fluid through the recirculation opening, or to provide a positive pressure on the recirculation opening. The sensor module detects the optical characteristic of the waste fluid in the detection window, and generates and transmits the optical characteristic signal to the controller. The controller determines the volume of blood loss in the manner previously described.

The fluid path may include the waste fluid being directed through the recirculation opening, the first leg of the trunk, the detection window, the second leg of the trunk, and the outlet opening. The cartridge may include an inner housing coupled to or otherwise engaging the trunk. The inner housing may be at least partially disposed within the trunk and include features contoured to complementary features of the trunk. The inner housing may include an inner first leg disposed within the first leg of the trunk, and an inner second leg disposed within the second leg of the trunk. The first seal may be coupled to the inner second leg, and the second seal may be coupled to the inner first leg. The first seal may be compressed between the inner second leg and the second leg to cover one of the outlet opening and the recirculation opening, and the second seal may be compressed between the inner first leg and the first leg to cover the other one of the outlet opening and the recirculation opening. The inner housing may further include a barrier separating an intake track and an exhaust track so as to constrain the fluid path towards and away from the detection window, respectively. The cartridge may also include a filter element disposed within the exhaust track such that the waste material being directed through the fluid path is not filtered prior to encountering the detection window.

The cartridge housing may include opposing flat faces that are parallel and larger than opposing outer sides such that the cartridge is a cassette in form. The void may extend inwardly for at least half of a length of each of the first leg and the second leg such that the cartridge housing is U-shaped or H-shaped. The void may be sized and shaped to receive the sensor module.

At least one of the first leg and the second leg may define the detection window. The ridge may extend into the void from at least one of the body portion, the first leg, and the second leg.

The spine of the cartridge housing may define the detection window. The opposing outer sides of the spine may be spaced apart from one another to define a channel in fluid communication with an interior of the body portion or the first leg. The arm may define the detection window by including opposing outer sides spaced apart from one another to define a channel in fluid communication with an interior of the body portion or the first leg. The first leg may include a barrier coupled to the rim to separate the recirculation opening and the outlet opening within the rim. Geometries internal to the first leg may be provided to maintain separation of the intake track and the exhaust track within the first leg, at least for a sufficient distance for the waste fluid to be detected by the sensor module.

The cartridge may also include a radiofrequency identification (RFID) tag including memory storing data. The RFID tag is configured to be detected by and transmit the data to a RFID reader coupled to the cartridge receiver or another suitable component of the medical waste collection system. The data may be indicative of compatibility of the cartridge with the medical waste collection system. The memory of the RFID tag may store calibration data for the emitters and/or the sensors. The data may be instructions for operating the medical waste collection system in a particular manner with the cartridge removably coupled to the cartridge receiver. The controller may receive the data from the RFID reader, and operate the pump and actuate valves to recirculate the waste fluid from the waste volume through the fluid path. The controller may receive the data from the RFID reader, activate the sensor module to perform the QBL analysis, and display output from the QBL analysis on the control panel.

The sensor module may be coupled to or integrated with the cartridge itself as opposed to the sensor housing. The emitters and/or the sensors may be coupled to the cartridge housing, the channel, or another suitable structure of the cartridge so as to be positioned adjacent or opposite the detection window. The sensor module disposed on the cartridge may be powered by a battery, or electrical contacts on the cartridge may engage complementary electrical contacts of the sensor housing to establish a power circuit. The sensor module may communicate data with the controller by a wireless communications module coupled to the cartridge housing, or a data coupler on the cartridge may engage complementary data coupler of the sensor housing to establish a data circuit. The sensor module may be removably couplable with the cartridge prior to or after insertion of the cartridge into the cartridge receiver. The cartridge receiver may define an inlet aperture and an outlet aperture of the fluid path, and an orientation feature configured to engage a complementary orientation feature of the cartridge. The cartridge receiver and the cartridge may include components to provide for selective locking of the cartridge once removably coupled with the cartridge receiver. The cartridge receiver may include an actuator configured to receive an input to move at least one component of the cartridge receiver between two positions to selective engage a complimentary geometry of the cartridge.

The cartridge housing of the cartridge may include an outer diameter complementary to an inner diameter of the cartridge opening. The cartridge may include a gasket coupled to the outer diameter of the cartridge housing and configured to seal the cartridge opening when the cartridge is removably coupled with the cartridge receiver. The cartridge housing may include a head separating two portions of the cartridge. Extending from the head are the shoulders defining the bores that form a portion of the fluid path. The bores may be arranged in parallel and positioned in a side-by-side relationship complementary to the inlet aperture and the outlet aperture of the cartridge receiver. The shoulders may define rims configured to be arranged to form a face seal with an inner surface of the cartridge receiver. The rims may define grooves, and gaskets may be disposed in the grooves to facilitate the face seal.

The detection window may be defined by a spine including the opposing outer sides. At least the detection window of the spine is optically clear, and an entirety of the cartridge housing may be optically clear. The opposing outer sides of the spine may facilitate a friction fit with complementary opposing inner sides of the sensor housing. At least the opposing inner sides of the sensor housing may be formed from a resilient material to facilitate the friction fit with the spine of the cartridge. The emitters and the sensors are coupled to the opposing inner sides of the sensor housing so as to be positioned adjacent or opposite the detection window. The spine and/or the head may form the handle of the cartridge to facilitate the cartridge being inserted into and removed from the cartridge receiver. The sensor housing may be coupled to the cartridge prior to insertion, and thereafter itself provide a grip to manipulate the sensor-cartridge assembly for insertion into the cartridge receiver. The sensor housing may include a dongle integrated with or removably coupled to a data and power port on the chassis of the medical waste collection system.

The cartridge may include the channel or tube, but otherwise be devoid of a discrete housing supporting the cartridge. The shoulders may extend from the spine to form a U-shaped or H-shaped cartridge. The shoulders define the bores, and the gaskets may be coupled near ends of the shoulders. The ends of the shoulders may have an outer diameter configured to be slidably and snugly inserted into a respective one of the inlet aperture and the outlet aperture of the cartridge receiver for the gaskets to form a face seal with the inner surface of the cartridge receiver.

The sensor housing may include at least one strut extending between the opposing inner sides so as to permit the sensor housing to define a sensor window. The sensor window may be elongate and contoured to the cartridge. The cartridge is removably inserted into the cartridge receiver, and the waste fluid may be visible through the sensor window. At least one brace or clamp may be provided to secure the sensor-cartridge assembly in the cartridge receiver. The waste fluid being circulated through the fluid path may be visualized without requiring removal of the sensor housing.

The cartridge receiver may define an outport in selective fluid communication between a first or upper waste container and a second or lower waste container. A motor may be coupled to a valve to establish the selective fluid communication between the upper waste container and the lower waste container. A recirculation mode includes operating the pump to draw the waste fluid from the waste volume and through the cartridge to be recollected in the waste volume. The transferring mode includes operating the motor to draw the waste fluid from the waste volume and through the outport to be collected in the lower waste container. Another implementation provides for at least the recirculation mode, the transferring mode, and a closed mode. The recirculation mode includes the pump to draw the waste fluid from the waste volume and through the cartridge to be recollected in the waste volume. The transferring mode includes operating the motor to position the valve to transfer the waste fluid from the waste volume through the outport to be collected in the lower waste container. The closed mode includes operating the motor to close the valve in which the waste fluid collects within the waste volume without performing QBL analysis. The sensor module may be coupled to or near the outport such that the emitters and the sensors are arranged to detect the waste fluid being transferred from the upper waste container to the lower waste container. The QBL analysis may be performed simultaneous with the emptying of the upper waste container.

The cartridge may define a fluid reservoir and includes means for drawing the waste fluid from waste container to within the fluid reservoir to facilitate the QBL analysis. The cartridge may include an actuator configured to be actuated between a closed position in which the waste fluid is not permitted to flow between the waste volume and the fluid reservoir, and an open position in which the waste fluid is permitted to flow between the waste volume and the fluid reservoir. Actuation of the actuator may itself urge the fluid to be drawn from the waste volume and into the fluid reservoir. The actuator may be pivotably coupled to the cartridge housing and include a hub, and a plunger coupled to the hub. The plunger may be arcuate and include a curvature approximate to the radius of the cartridge housing. The hub may be mounted near a center of the cartridge housing so as to pivot about its center and move the plunger in an arcuate manner. The cartridge may include a motor coupled to the cartridge housing and configured to pivot the hub relative to the cartridge housing between the open and closed positions. The motor may be controller by the controller, and an input to the control panel may operate the cartridge to be moved between the open and closed positions. The hub of the actuator may further include at least one control surface configured to receive a manual input to move the actuator between the open and closed positions. With the actuator being moved from the closed position to the open position, the plunger is moved arcuately to expose the fluid reservoir and the inlet, and the waste fluid may be drawn into the fluid reservoir. The actuator may be actuated to draw the waste fluid under the influence of a vacuum from the waste volume, through the inlet, and into the fluid reservoir. The sensor module detects the optical characteristic of the waste fluid in the detection window. The actuator may be further actuated for the plunger to circulate the waste fluid from the fluid reservoir, through the outlet, to be recollected in the waste volume. The plunger may itself or include a sealing member to slidably or frictionally remove or clean residual amounts of the waste fluid from the detection window.

A tank may define the fluid reservoir. The tank may be integrated with or removably coupled to the chassis, or may be removably coupled to a cradle that is mounted to the chassis. Alternatively, the tank may be supported atop a stand that includes wheels. The tank may include a base, and a lid coupled to the base to define the fluid reservoir. The base or the lid may be at least partially or entirely clear to define the detection window. A removable or openable cover may be coupled to or integrated with the tank so as to limit visibility. The base may include the spine extending from a sidewall of the tank. The tank may include the agitator. The base or the lid may include at least one inlet fitting, and at least one outlet fitting. The inlet fitting and the outlet fitting are configured to be removably coupled to a suction tube. The suction tube coupled to the outlet fitting may further be coupled to the inlet fitting of the manifold removably inserted into the manifold receiver. The vacuum provided by the vacuum source draws the waste fluid through the distal suction tube to be collected in the fluid reservoir. In another implementation, the suction tube coupled to the outlet fitting may further be coupled to a separate vacuum source, such as a vacuum system integrated with the operating theatre, such that the tank is a standalone unit. With the waste fluid disposed within the detection window, the sensor module detects the optical characteristic of the waste fluid in the detection window. The controller determines the volume of blood loss in the fluid reservoir, and the blood loss may be displayed on a display.

In certain implementations, the QBL analysis may be performed by coupling the sensor module to the manifold, and recirculating the waste fluid from the waste volume and through the manifold. The suction tube is coupled to the inlet fitting, and the vacuum from the vacuum source draws the waste fluid from the surgical field through the suction tube and into the manifold. The waste fluid is drawn through an initial or bypass flow path in which it is not directed through the detection window. The bypass flow path includes the waste fluid being directed through a filter element disposed within the trunk, and an outlet opening. Once exiting the manifold through the outlet opening, a container inflow path includes the waste fluid being drawn through an intake of the manifold receiver and into the waste container. The fluid measurement system is configured to generate the level signal representative of a fluid level of the waste fluid collected within the waste container, and transmit the waste level signal to the controller. The pump may be operated to circulate the waste fluid in a container outflow path from the waste volume through a discharge of the manifold receiver and into a recirculation opening defined by the trunk of the manifold.

The waste fluid is directed in a recirculation flow path towards the sensor module coupled to a head of the manifold, and further directed through the detection window positioned between the emitters and the sensors of the sensor module. The waste fluid may bypass the filter element as it is recirculated towards the sensor module. The sensor module detects the optical characteristic of the waste fluid in the detection window, and generates and transmits the optical characteristic signal to the controller. The controller determines the blood concentration in the waste fluid passing through the manifold. The flow path may be rejoined with the bypass path at a position proximal to the sensor module to again be directed through the filter element and the outlet opening. The manifold may include an RFID tag including memory storing data indicative of compatibility of the manifold, and/or instructions for operating the medical waste collection system in a particular manner with the manifold for facilitating the QBL analysis.

Similar to certain implementations of the cartridge, the outlet opening of the manifold may be defined in a first leg of the trunk, and the recirculation opening is defined in a second leg of the trunk. The outlet opening may be positioned below the recirculation opening when the manifold is oriented for insertion into the manifold opening of the manifold receiver. The first leg and the second leg may be spaced apart from one another to at least partially define a void sized to receive a depth element of an inlet mechanism. The manifold may be configured to be inserted into and removed from the manifold receiver in a proximal direction and a distal direction. The inlet mechanism is configured to move in the proximal and distal directions opposite of a direction of movement of the manifold to prevent fluid communication between the manifold and the vacuum source until the manifold is in a fully inserted operative position.

The inlet mechanism may define a first pathway and a second pathway configured to establish the fluid communication between the manifold and the waste container. The first pathway may be arranged in fluid communication with the outlet opening of the manifold. and the second pathway is configured to be arranged in fluid communication with the recirculation opening of the manifold. The first pathway may define a portion of the container inflow path, and the second pathway may define a portion of the container outflow path. The vacuum from the vacuum source through the first pathway draws the waste fluid from the surgical field through the suction tube and into the manifold. The waste fluid is drawn through the first leg, the outlet opening, and the first pathway to be collected in the waste volume. The fluid measurement system is configured to generate and transmit the waste level signal to the controller. The vacuum source or the pump may be operated to circulate the waste fluid from the waste volume through the second pathway and into recirculation opening defined by the second leg of the trunk of the manifold.

The manifold may include a second inlet fitting configured to be coupled with a suction tube that itself is coupled to discharge port of the waste container. The second inlet fitting may be disposed on the head and positioned below the first inlet fitting. The second inlet fitting is configured to receive the waste fluid being recirculated by the pump. The sensor module may be positioned proximal to the second inlet fitting and on opposing sides of the detection window. The second inlet fitting may provide the bypass flow path, or arranged to provide for the QBL analysis through two suction tubes simultaneously.

### BRIEF DESCRIPTION OF THE DRAWINGS

Advantages of the present disclosure will be readily appreciated as the same becomes better understood by reference to the following detailed description when considered in connection with the accompanying drawings.

FIG. 1 is a perspective view of a medical waste collection system with a manifold removably inserted into a receiver of the medical waste collection system. A cartridge is configured to be removably inserted into a cartridge receiver of the medical waste collection system.

FIG. 2 is perspective view of a portion of the medical waste collection system of FIG. 1 in which an outer casing is removed to show the cartridge receiver. A portion of the waste container is removed to show an agitator disposed within the waste container.

FIG. 3 is a schematic representation of certain subsystems of the medical waste collection system of FIG. 1. An inlet mechanism of the manifold receiver defines a pathway providing a recirculation path between the waste volume and the manifold removably inserted into the manifold receiver.

FIG. 4A is a representation of an arrangement of the medical waste collection system in which the cartridge is configured to be removably coupled to the cartridge receiver, and a sensor housing is configured to be removably coupled to the cartridge.

FIG. 4B is a front perspective view of a cartridge configured to be removably inserted within a cartridge opening of the cartridge receiver, and a sensor housing configured to be removably coupled to the cartridge.

FIG. 4C is a rear perspective view of the cartridge and the sensor housing of FIG. 4B.

FIG. 4D is a representation of an arrangement of the cartridge in which an inner housing defines an intake track and an exhaust track to provide a fluid path through the cartridge.

FIG. 4E is a plan view of another cartridge configured to be removably inserted within the opening of the cartridge receiver and including a cartridge housing defining a void configured to receive the sensor housing within the cartridge receiver.

FIG. 4F is a sectional elevation view of the cartridge and the sensor housing of FIG. 4E taken along section 4F-4F.

FIG. 4G is a top perspective view of still another cartridge configured to be removably inserted within the cartridge opening of the cartridge receiver and including a spine defining a detection window.

FIG. 4H is an elevation view of the cartridge and the sensor housing of FIG. 4G.

FIGS. 5A and 5B are representations of another arrangement of the medical waste collection system in which the cartridge is configured to be removably coupled to the cartridge receiver, and the sensor housing is configured to be removably coupled to the cartridge.

FIG. 6 is a representation of still another arrangement of the medical waste collection system in which the cartridge is configured to be removably coupled to the cartridge receiver, and the sensor housing is configured to be removably coupled to the cartridge.

FIGS. 7A-7C are representations of an arrangement of the medical waste collection system in which the cartridge defines a fluid reservoir and includes means for drawing the waste fluid from the waste volume into the fluid reservoir.

FIGS. 8A-8C are representations of another arrangement of the medical waste collection system in which a tank defines the fluid reservoir.

FIG. 9 is a perspective view of an arrangement in which the tank defining the fluid reservoir is supported atop a stand.

FIG. 10 is a representation of an arrangement of the medical waste collection system in which the waste fluid is recirculated through the manifold removably inserted into the manifold receiver.

FIG. 11A is a rear perspective view of another cartridge configured to be removably inserted within the cartridge opening of the cartridge receiver and including an arm defining the detection window.

FIG. 11B is a rear perspective view of another cartridge configured to be removably inserted within the cartridge opening of the cartridge receiver and including the spine defining the detection window.

FIG. 12 is a perspective view of another manifold through which the waste fluid is recirculated.

FIGS. 13A-13G are perspective views of the sensor housing removably coupled to the manifold.

### DETAILED DESCRIPTION

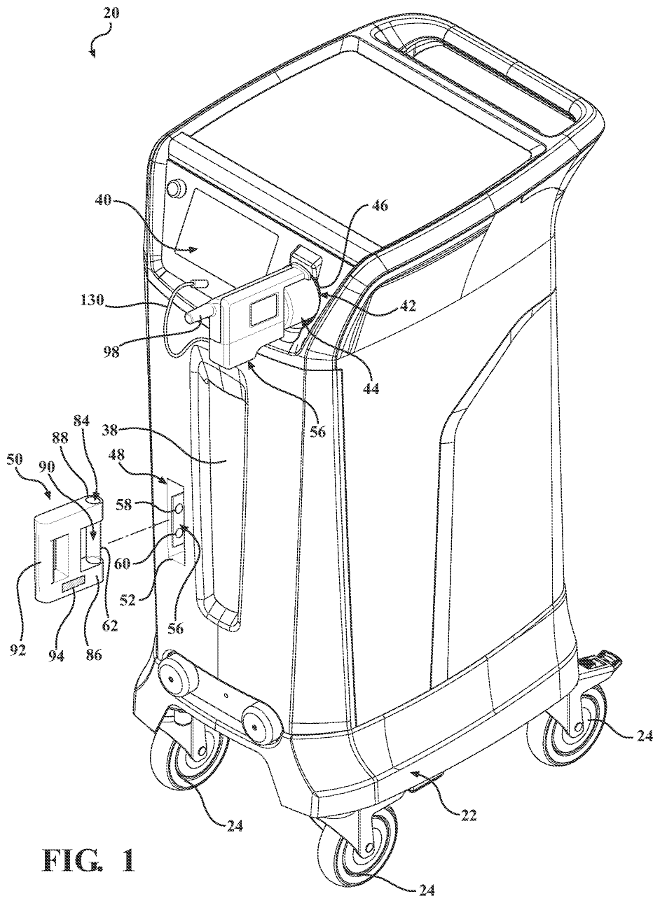

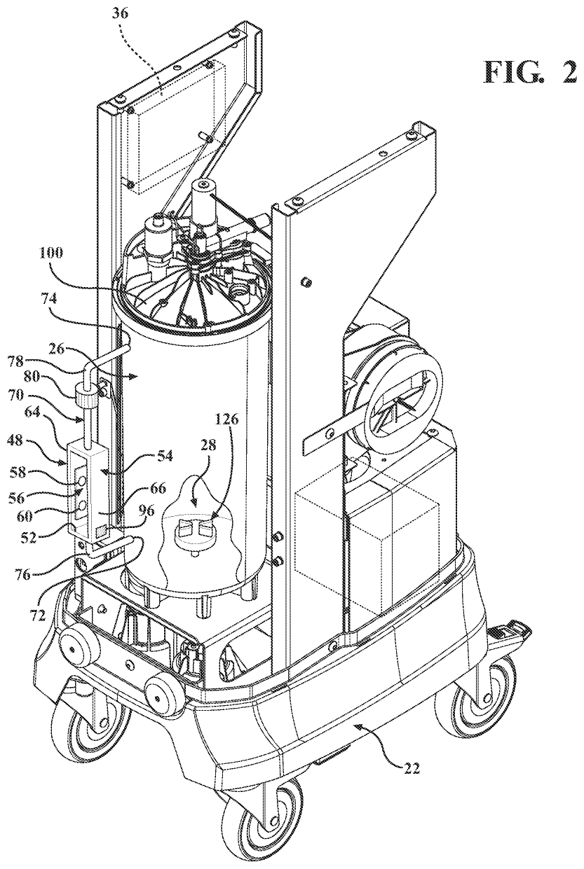

FIGS. 1 and 2 show a medical waste collection system 20 for collecting the waste material generated during medical procedures, and more particularly surgical procedures in which suctioning the waste material may be indicated. The waste material may include smoke, body tissues, and waste fluid such as bodily fluids and irrigation liquids. The medical waste collection system 20 collects and stores the waste fluid until it is necessary or desired to off-load and dispose of the waste fluid. The medical waste collection system 20 may be transported to and operably coupled with a docking station through which the waste fluid is emptied. The docking station may assume any suitable form, for example, as disclosed in commonly-owned U.S. Pat. No. 7,621,898, issued Nov. 24, 2009, the entire contents of which are hereby incorporated by reference.

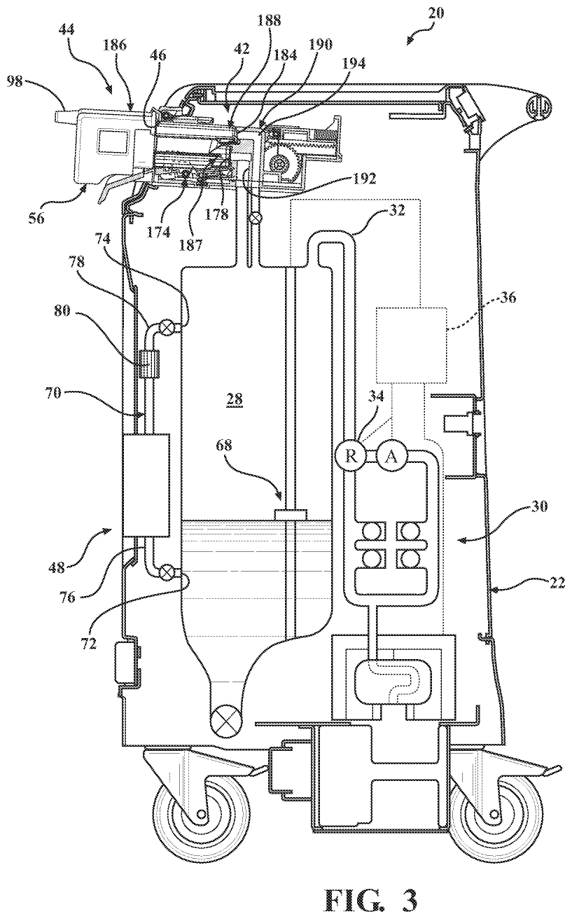

The medical waste collection system 20 may include a chassis 22 and wheels 24 for moving the chassis 22 along a floor surface within a medical facility. The medical waste collection system 20 includes at least one waste container 26 defining a waste volume 28 for collecting and storing the waste fluid. With concurrent reference to FIG. 3, a vacuum source 30 may be supported on the chassis 22 and configured to draw suction on the waste volume 28 through one or more internal lines or conduits 32. The vacuum source 30 may also include a vacuum regulator 34 supported on the chassis 22 and in fluid communication with the vacuum source 30 and the waste container 26. The vacuum regulator 34 is configured to regulate the vacuum level drawn by the vacuum source 30 on the waste container 26. At least one manifold receiver 42 supported on the chassis 22 defines a manifold opening 46 sized to removably receive a manifold 44 to be described. During operation of the medical waste collection system 20, a suction path is established from suction tube(s) to the waste volume 28 through the manifold 44 removably inserted into the manifold receiver 42. The vacuum generated by the vacuum source 30 is drawn on the suction tube, and the waste fluid at the surgical site is drawn through the manifold 44 to be collected in the waste container 26.

The medical waste collection system 20 further includes a fluid measurement system 68 disposed within the waste container 26 and in electronic communication with a controller 36. One suitable fluid measuring system 68 is disclosed the aforementioned U.S. Pat. No. 7,612,898. The fluid measurement system 68 may include a float element configured to float upon the waste fluid and move along a sensor rod. An interrogating signal is transmitted along the sensor rod, and a return signal is detected based on the position of the float clement along the sensor rod. The fluid measurement system 68 is configured to generate a level signal representative of a fluid level of the waste fluid collected within the waste container 26, and transmit the waste level signal to the controller 36. The controller 36 is configured to determine a volume of the waste fluid based on the waste level signal. The determined volume of the waste volume and a determined blood concentration is utilized in the QBL analysis to be described to determine a volume of blood loss. Additional or alternative means for measuring and determining the volume of the waste fluid, or a portion thereof, are contemplated. In one example, a flow sensor may be provided and configured to measure volumetric flow. Based on the volumetric flow over a known period of time, the volume may be determined. The volumetric flow may be fixed, variable, averaged, or the like. In another example, a pump 80 to be described may in communication with the controller 36 and configured to generate a signal representative of the volume of the waste fluid. For example, based on a known flow capacity, the pump 80 may include a sensor to index turns of the pump rotor or measure the speed of the pump, from which the volume may be determined. The pump 80 may provide the signal to the controller 36.

A control panel 40 disposed on the chassis 22 is in communication with the controller 36. The user may select on the control panel 40 operating parameters of the medical waste collection system 20, such as the vacuum level drawn on the waste container 26. The controller 36 is configured to control the vacuum source 30 and/or the vacuum regulator 34 to operate the medical waste collection system 20 based on the operating parameters. The control panel 40 may further display the operating parameters and other information, such as the volume of blood loss as determined by the controller 36.

A front of the chassis 22 may define a window 38 to permit a user to view the contents within the waste volume 28 of the waste container 26. Particularly where the waste material includes bodily fluids such as blood and non-blood fluids, visualizing the contents of the waste container 26 may provide for qualitative assessment of the extent of blood loss. The qualitative analysis may be in addition to the QBL analysis. For example, the user may visually monitor the color of a mixture of the blood and non-blood fluids through the window 38, and should the color become too reddish in color indicative of excessive blood loss, the user may choose to view the control panel 40 that displays the QBL analysis in real-time.

Suitable construction and operation of the aforementioned and additional subsystems of the medical waste collection system 20 may be disclosed in commonly-owned United States Patent Publication No. 2005/0171495, published Aug. 4, 2005, International Publication No. WO 2007/070570, published Jun. 21, 2007, International Publication No. WO 2014/066337, published May 1, 2014, International Publication No. WO 2017/112684, published Jun. 29, 2017, and International Publication No. WO 2020/210763, published Oct. 15, 2020, the entire contents of which are hereby incorporated by reference.

The medical waste collection system 20 includes a cartridge receiver 48 configured to removably receive a cartridge 50 to facilitate the QBL analysis. The cartridge receiver 48 may include a cartridge opening 52 defined by a sensor housing 54 or another housing. A sensor module 56 is coupled to the sensor housing 54 and includes at least one emitter 58 and at least one sensor 60. The emitters 58 are configured to emit energy, and the sensors 60 are configured to detect the emitted energy. An exemplary implementation utilizes light energy with the emitters 58 being light emitting diodes (LEDs) and the sensors 60 being photodetectors. The sensors 60 detect the emitted light, and more particularly the light after being transmitted or scattered through the waste fluid. A first emitter may be an infrared LED, and the second emitter may be a visible-light LED, such as a green LED. The infrared LED may be configured to emit light having a wavelength approximately in the range of 700 nanometers (nm) to 1000 nm, and more particularly within the range of 750 nm to 850 nm, and even more particularly within the range of 770 nm to 810 nm. The visible-light LED may be configured to emit light having a wavelength approximately in the range of 400 nm to 600 nm, and more particularly within the range of 550 nm to 600 nm, and even more particularly within the range of 570 nm to 580 nm. A first sensor may detect transmitted light and scattered light from the first emitter, and a second sensor may detect transmitted light and scattered light from the second emitter. The four measurements—two of the transmitted light and two of the scattered light—are values provided to the controller 36 (or another processor) to perform the QBL analysis. An alternative arrangement includes two of the emitters 58 and four of the sensors 60. Each of the four sensors 60 may be configured to detect one of the four measurements—two of the transmitted light and two of the scattered light. There may be more or less than two emitters 58 with the additional emitters configured to detect optical characteristics of non-blood fluids, such as amniotic fluid, bile, or the like. The emitters 58 and sensors 60 may be arranged in optical communication with the fluid path in any suitable configuration.

The detected intensity of the transmitted and/or the scattered light may be indicative of the transmissivity, opacity, and/or other physical property of the waste fluid. More particularly, a measurement of the infrared light absorbed by the waste fluid is determined by a reduction in transmitted light due to the presence of the blood within the waste fluid. In one implementation, a ratio of this absorbance of the infrared light to scattered light from the visible light is calculated and used to quantify the concentration of blood in the waste fluid.

The emitters 58 and the sensors 60 are configured to be positioned adjacent a fluid path 70, and more particularly opposite a detection window 62 defining a portion of the fluid path 70. In the implementation illustrated in FIG. 2, the sensor housing 54 includes a first sidewall 64, and a second sidewall 66 opposite the first sidewall 64 to define the cartridge opening 52 and a void sized to receive at least a portion of the cartridge 50. The cartridge 50 includes the detection window 62. FIG. 2 shows one of the emitters 58 and one of the sensors 60 coupled to the first sidewall 64. The other one of the emitters 58 and the other one of the sensors 60 (not shown) is coupled to the second sidewall 66. With the cartridge 50 removably inserted into the cartridge receiver 48, the detection window 62 of the cartridge 50 is positioned between the emitters 58 and the sensors 60.

The emitters 58 and the sensors 60 may be positioned adjacent to the fluid path in any suitable configuration. More than one of the emitters 58 and/or more than one of the sensors 60 may be coupled to the first sidewall 64 and/or the second sidewall 66 (or any other structure of the sensor housing 54 or the cartridge receiver 48). It is further contemplated that more than two emitters 58 and/or sensors 60 may be provided. In the example with two emitters 58 and four sensors 60, the emitters 58 may be coupled to the same or different sidewall 64, 66, and/or the sensors 60 may likewise be coupled to the same or different sidewall 64, 66.

Certain known suction systems include an optical detector coupled to the suction tube. Such in-line optical systems may fail to provide sufficient accuracy of the volume of blood loss, as there is a time lag between determined blood concentration values and a determined volume of the collected waste fluid. In other words, the instant where the blood may be optically sensed within the suction tube is not matched to the volume of the collected waste fluid in the waste container at that instant. This delay may be variable and based on any number of changing factors, including vacuum level, blood clotting, blood temperature, contaminants, the blood concentration itself, and the volume of waste fluid in the waste container, making predicative algorithmic corrections especially difficult. The medical waste collection system 20 overcomes such technical shortcomings by circulating (also referred to herein as recirculating) the waste fluid from the waste volume 28 to be analyzed for the QBL analysis. The waste fluid being recirculated already has been included in the determined volume of the waste fluid, thereby eliminating any time lag or mismatching with the determined blood concentration. With reference to FIGS. 2 and 3, the medical waste collection system 20 includes the fluid path 70 defining an inlet 72 and an outlet 74 each in fluid communication with the waste volume 28. For example, a first conduit 76 is coupled to an exterior of the waste container 26 to define the inlet 72, and a second conduit 78 is coupled to the exterior of the waste container 26 to define the outlet 74. The first conduit 76 and the second conduit 78 may define a portion of the fluid path 70. A pump 80 and valves are arranged in fluid communication with the waste volume 28 and configured to circulate the waste fluid from the waste volume 28 through the fluid path 70 to be recollected within the waste volume 28. The pump 80 may be coupled to the first or second conduits 76, 78 to define a portion of the fluid path 70. The pump 80 may be further configured to provide a positive or negative pressure to circulate the waste fluid from the waste volume 28 through the fluid path 70.

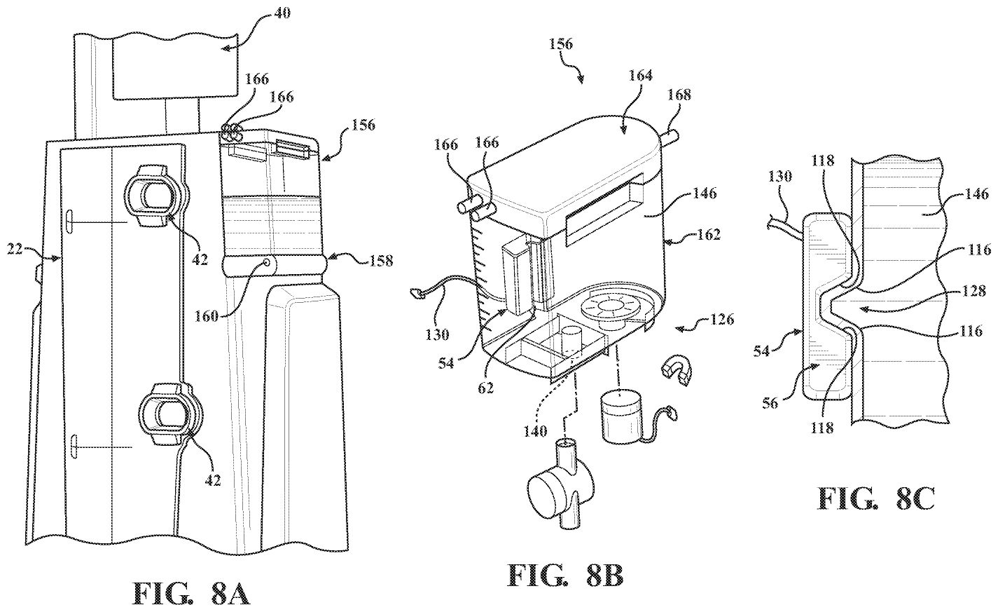

The medical waste collection system 20 may further include an agitator 126 disposed within the waste container 26. Certain points of the surgical procedure may be associated with more or less blood loss relative to the non-blood fluids, and thus the waste fluid may collect within the waste volume 28 in a non-homogeneous manner. As a result, portions of the collected waste fluid being recirculated through the fluid path 70 may be under-or over-representative of the actual blood concentration of the waste fluid within the waste container 26. The agitator 126 is configured to facilitate homogenization of the waste fluid within the waste volume 28. The agitator 126 includes an impeller coupled to a motor (not shown) through a mechanical shaft or magnetic drive (see FIG. 8B), and the impeller mixes the waste fluid in the waste container 26. The agitator 126 may be in communication with the controller 36, and the controller 36 may selectively operate the agitator 126 based on any number of operating parameters. In one example, the controller 36 may toggle off and on the agitator 126 for fixed or varied intervals. In a second example, the instantaneous blood concentration values may be too erratic and indicative of non-homogeneity. Therefore, should the instantaneous blood concentration values deviate from one another greater than a predetermined threshold for a certain period of time, the controller 36 may initiate operation or speed up operation of the agitator 126. In a third example, the controller 36 may operate or speed up the agitator 126 during any periods in which the pump 80 is being operated indicative of a QBL operating mode. In a fourth example, the user may also provide an input to the control panel 40 to selectively operate the agitator 126 as needed. For example, the user may visually observe through the window 38 that pockets or portions of the waste volume 28 are appreciably redder than others, after which the input may be provided to operate the agitator 126 to mix the waste fluid.

It should also be understood that recirculating the waste fluid through the fluid path 70 may itself facilitate homogenization of the waste fluid within the waste volume 28. In other words, lesser homogeneous portions of the waste fluid are drawn through the fluid path 70 and the pump 80, during which turbulent or laminar flow within the fluid path 70 and/or through the pump 80 may mix the waste fluid. Redepositing of the waste fluid within the waste container 26, perhaps from above the fluid level, may provide a further agitative effect.

Likewise, the presence of air may affect the optical characteristics of the waste fluid being circulated through the fluid path 70. The air may transmit and scatter light in a manner different than blood such that the emitted and scattered light detected by the sensors 60 may not accurately represent the optical characteristics of the waste fluid. In certain arrangements, the inlet 72 may be located below the outlet 74 such that the pump 80 is configured to pump the waste fluid through the fluid path 70 against gravity. The waste container 26 may be prefilled with the waste fluid or another fluid, such as water, to a fluid level above the inlet 72 of the fluid path 70 prior to circulating with the pump 80 the waste fluid through the fluid path 70 or activating the sensor module 56. The prefilled fluid level may be coordinated by the fluid measuring system 68 and the controller 36. The arrangements may bleed the air from the fluid path 70 prior to or concurrent with commencing the QBL analysis. Additionally or alternatively, the waste fluid may be diluted with the waste fluid or another fluid, such as water, such that the blood concentration is below a predetermined blood concentration that itself may be based on optical capabilities of or optimal conditions for the sensors 60.

The sensor module 56 is arranged to detect the optical characteristic (e.g., the transmissivity, opacity, and/or other physical property) of the waste fluid indicative of the blood concentration of the waste fluid being circulated through the fluid path 70. In the illustrated implementation of FIGS. 2 and 3, the arrangement includes the sensor housing 54 being coupled to the first or second conduits 76, 78 with the void defined by the sensor housing 54 defining a portion of the fluid path 70. The sensor module 56 is coupled to the sensor housing 54 in the manner previously explained such that the emitters 58 and the sensors 60 are positioned adjacent or opposite the fluid path 70.

It is understood that the waste fluid generally should not directly contact the emitters 58, the sensors 60, and other electronic components of the sensor module 56. As such, an optically clear channel or tube that includes the detection window 62 overcomes such concerns while facilitating the QBL analysis. However, the waste fluid, including the blood in the waste fluid, may foul the clear channel or tube over time. In particular, the transparency of the clear channel or tube may degrade such that the emitted and scattered light detected by the sensors 60 may not accurately represent optical characteristics of the waste fluid. It is contemplated that the medical waste collection system 20 may include a cleaning system (not shown) configured to flush the fluid path 70 with a detergent or other fluid to restore the transparency of the clear channel or tube.

In exemplary implementations, the medical waste collection system 20 includes the cartridge 50 that itself includes the detection window 62. The cartridge 50 is removably inserted into the cartridge receiver 48, and may be disposable after a single use to obviate the aforementioned fouling concerns. The cartridge 50 includes a cartridge housing 82 sized to be inserted through the cartridge opening 52 and at least partially disposed within the sensor housing 54. The cartridge housing 82 defines a passageway 84 configured to form or complete the fluid path 70 with the cartridge 50 removably coupled to the cartridge receiver 48. In one example, FIG. 1 shows the cartridge housing 82 including shoulders 86 each defining a bore 88 (one shown). The channel 90 may be coupled to the shoulders 86 to define the passageway 84 in fluid communication with the bores 88. The channel 90 may itself define the detection window 62 that is configured to be positioned between the emitters 58 and the sensors 60 when the cartridge housing 82 is at least partially disposed within the void. The channel 90 may be a tube as shown or a member with another suitable cross section, or alternatively the cartridge housing 82 may define the detection window 62 through cutouts or other geometries formed within the cartridge housing 82. In other words, the detection window 62 may be a discrete component from or integrally formed with the cartridge housing 82.

The cartridge 50 may include a handle 92 with the shoulders 86 extending from the handle 92. The handle 92 may be positioned exterior to casing of the chassis 22 to facilitate the cartridge 50 being inserted into and removed from the cartridge receiver 48, respectively. Further, the cartridge 50 may include retention features (not shown) configured to releasably engage complementary retention features of the cartridge receiver 48. For example, the retention features may form an interference fit or friction fit, or alternatively may be a movable structure configured to be actuated between engaged and disengaged positions. The actuation between the engaged and disengaged positions may be provided through a mechanical input on the chassis 22, or an input on the control panel 40 in which the controller 36 electronically moves the structure to the disengaged position to permit removal of the cartridge 50.

The cartridge 50 may also include an RFID tag 94 including memory storing data. The RFID tag 94 is configured to be detected by and transmit the data to a RFID reader 96 coupled to the cartridge receiver 48 or another suitable component of the medical waste collection system 20. The data may be indicative of compatibility of the cartridge 50 with the medical waste collection system 20. The memory of the RFID tag 94 may store calibration data for the emitters 58 and/or the sensors 60. Additionally or alternatively, the data may be instructions for operating the medical waste collection system 20 in a particular manner with the cartridge 50 removably coupled to the cartridge receiver 48. For example, the controller 36 may receive the data from the RFID reader 96, and operate the pump 80 and actuate valves to recirculate the waste fluid from the waste volume 28 through the fluid path 70. For another example, the controller 36 may receive the data from the RFID reader 96, activate the sensor module 56 to perform the QBL analysis, and display output from the QBL analysis on the control panel 40. Should no such data be received by the controller 36, the controller 36 may determine that no cartridge 50 is present and operate the medical waste collection system 20 with different operating parameters.

In certain implementations, the sensor module 56 may be coupled to or integrated with the cartridge 50 itself as opposed to the sensor housing 54. More particularly, the emitters 58 and/or the sensors 60 may be coupled to the cartridge housing 82, the channel 90, or another suitable structure of the cartridge 50 so as to be positioned adjacent or opposite the detection window 62 the manner previously described. The sensor module 56 disposed on the cartridge 50 may be powered by a battery, or alternatively electrical contacts on the cartridge 50 may engage complementary electrical contacts of the sensor housing 54 to establish a power circuit. Further, the sensor module 56 may communicate data with the controller 36 by a wireless communications module coupled to the cartridge housing 82, or alternatively a data coupler on the cartridge 50 may engage complementary data coupler of the sensor housing 54 to establish a data circuit. It is contemplated that the cartridge 50 may itself include a controller (not shown) in communication with the sensor module 56 and configured to perform any of the aforementioned functions of the medical waste collection system 20 related to the QBL analysis.

The QBL analysis performed by the medical waste collection system 20 is described with continued reference to FIGS. 1-3. The manifold 44 is removably inserted into the manifold receiver 42, and the suction tube (not shown) is coupled to an inlet fitting 98 of the manifold 44. The cartridge 50 is removably inserted into the cartridge receiver 48. A first input is provided to the control panel 40 to operate the vacuum source 30 to establish the suction path from the suction tube and through the manifold 44 and the manifold receiver 42 to be collected in the waste volume 28 of the waste container 26. The fluid measurement system 68 provides the waste level signal to the controller 36, and the controller 36 determines the waste volume of collected waste fluid in the waste container 26. Either with the first input or with another input to the control panel 40, or automatically based on the insertion of the cartridge 50, the controller 36 operates the pump 80 to circulate the waste fluid from the waste volume 28 through the fluid path 70 to be recollected in the waste volume 28. The sensor module 56 detects the optical characteristic of the waste fluid, and generates and transmits an optical characteristic signal to the controller 36. The controller 36 determines the blood concentration of the waste fluid based on the optical characteristic signal, and further determines the volume of blood loss based on the waste volume and the blood concentration. The volume of blood loss may be displayed on the control panel 40 or another display in communication with the medical waste collection system 20, such as a separate monitor or mobile device. The QBL analysis may be repeated continuously as desired so as to update the volume of blood loss in real-time. Should it be indicated to stop or restart the QBL analysis, the user need only provide a subsequent input to the control panel 40 for the controller 36 to operate the subsystems accordingly. Since the waste container 26 and the sensor module 56 are supported on the chassis 22 and in communication with the controller 36, the control panel 40 may provide all relevant information, obviating the need for the user to divert attention to observe separate fluid containers positioned in the operating theatre. Further, as mentioned, superior accuracy in the determined volume of blood loss is realized by recirculating the waste fluid after its initial collection within the waste container 26. Audible and/or visual alerts may be provided should the volume of blood loss exceed a predetermined or selected value. The volume of blood loss may also be uploaded to the patient electronic medical record (EMR) during or after the surgical procedure.

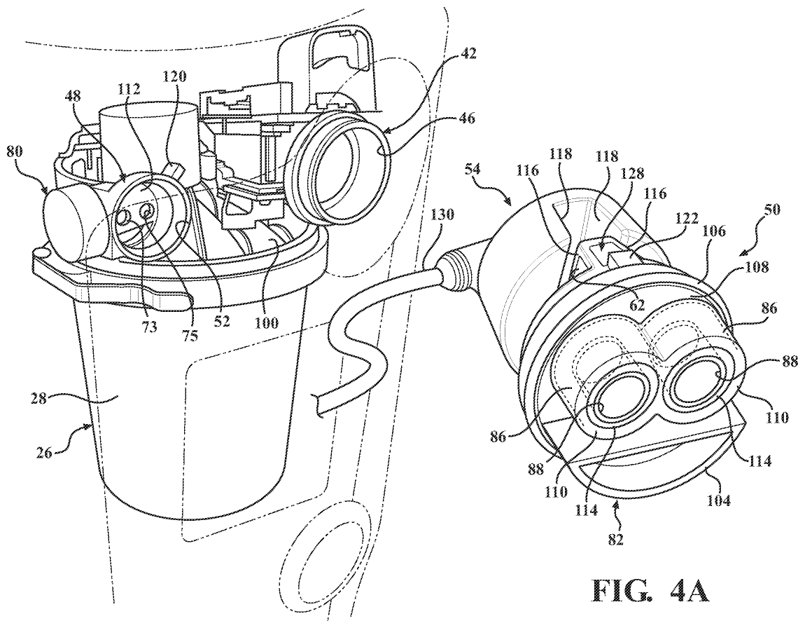

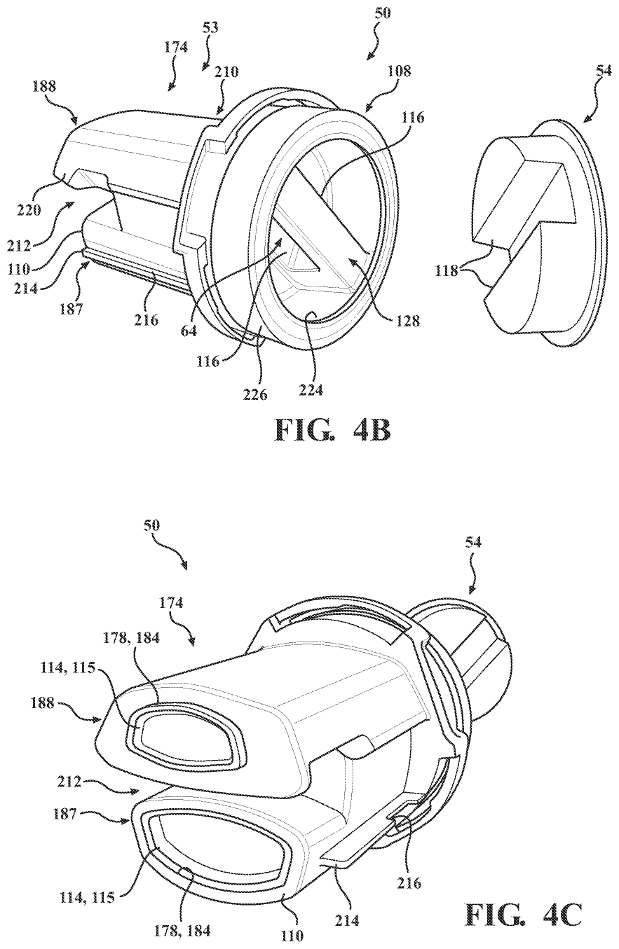

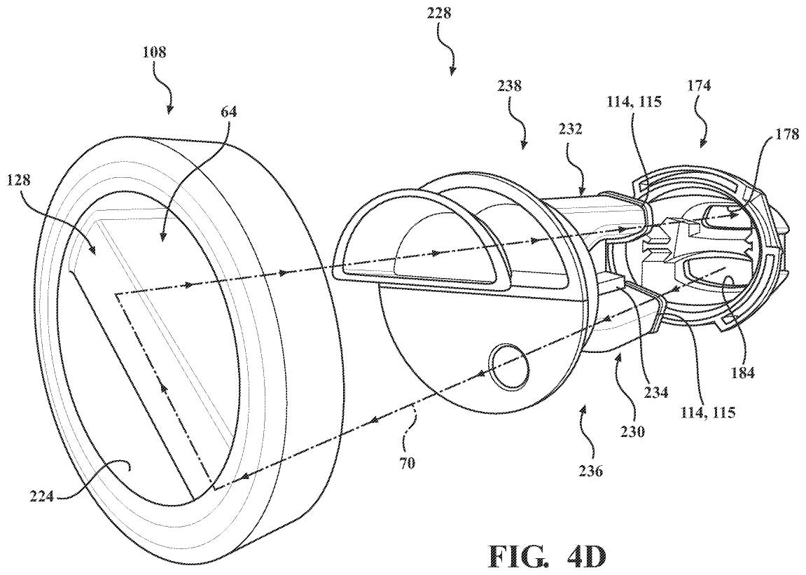

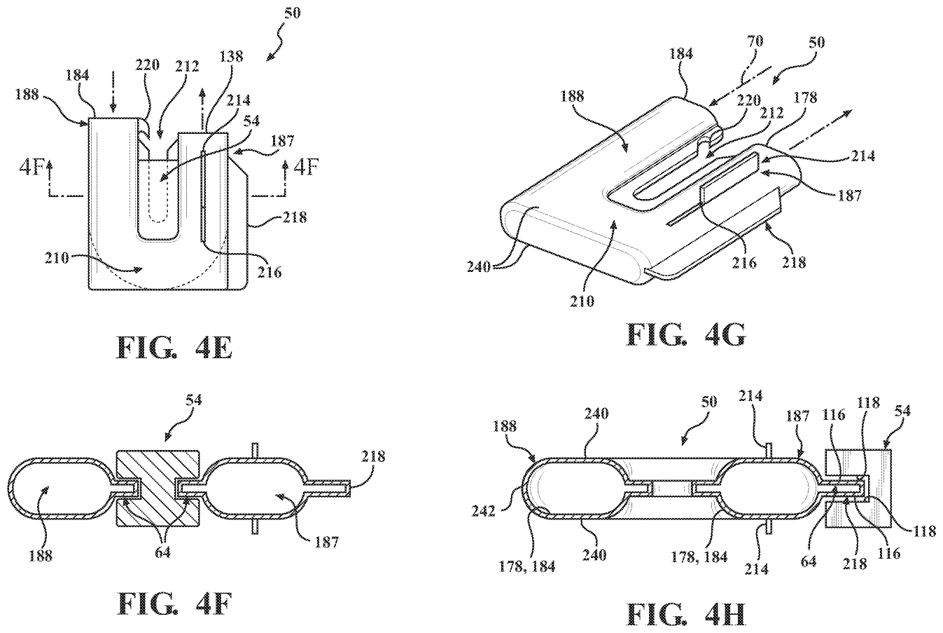

Referring now to FIG. 4A, another implementation of the cartridge 50 includes the sensor housing 54 being removably couplable with the cartridge 50 prior to or after insertion of the cartridge 50 into the cartridge receiver 48. The removable coupling of the sensor housing 54 with the cartridge 50 positions the sensor module 56 in opposing sides of the detection window 62 of the cartridge 50. A representative drawing of the waste container 26 is shown in which the cartridge receiver 48 is coupled to a lid or cap 100 of the waste container 26, and the pump 80 for the fluid path 70 is coupled to the cartridge receiver 48. The cartridge receiver 48 defines the cartridge opening 52, which is circular in the present implementation. The cartridge receiver 48 further defines an inlet aperture 73 and an outlet aperture 75 of the fluid path 70, and an orientation feature 102 configured to engage a complementary orientation feature 104 of the cartridge 50. The orientation feature 102 is shown as a semielliptical opening configured to receive a semielliptical protrusion forming the orientation feature 104 of the cartridge 50.

The cartridge housing 82 of the cartridge 50 includes an outer diameter complementary to an inner diameter of the cartridge opening 52. The cartridge 50 may include a gasket 106 coupled to the outer diameter of the cartridge housing 82 and configured to seal the cartridge opening 52 when the cartridge 50 is removably coupled with the cartridge receiver 48. For directional convention, the cartridge housing 82 includes a head 108 separating two portions of the cartridge 50. Extending from the head 108 in a first direction are the shoulders 86 defining the bores 88 that form a portion of the fluid path 70. Whereas the implementation of FIG. 2 shows the bores 88 being coaxially arranged and the channel 90 positioned therebetween, FIG. 4A shows the bores 88 being arranged parallel and positioned in a side-by-side relationship complementary to the inlet aperture 73 and the outlet aperture 75 of the cartridge receiver 48. The shoulders 86 may be cylinders with an outer circumference approximating a contact of circles as referred to in the geometric arts. The shoulders 86 define rims 110 configured to be arranged to form a face seal with an inner surface 112 of the cartridge receiver 48. The rims 110 may define grooves (not identified), and gaskets 114 may be disposed in the grooves to facilitate the face seal.

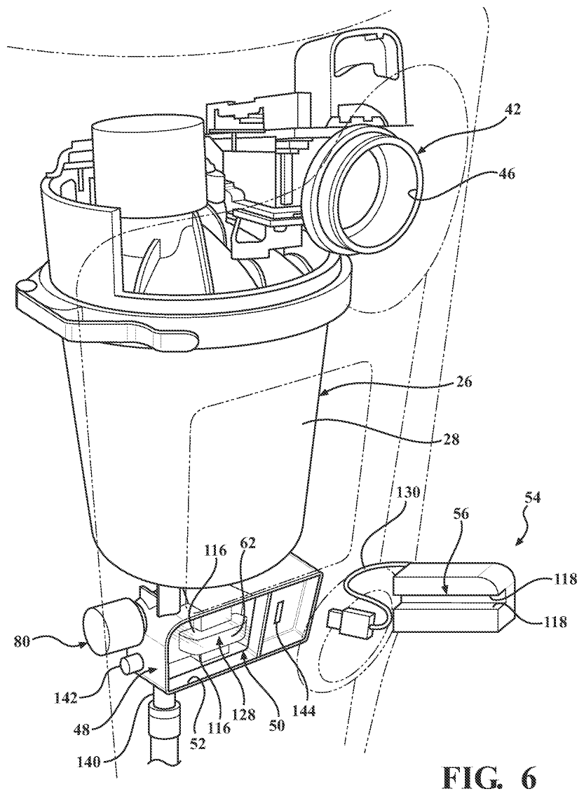

Extending from the head 108 in a second direction opposite the first direction is the detection window 62. The detection window 62 may be defined by a spine 128 including opposing outer sides 116. The opposing outer sides 116 may include a slight taper to facilitate a friction fit with complementary opposing inner sides 118 of the sensor housing 54. At least the opposing inner sides 118 of the sensor housing 54 may be formed from a resilient material to facilitate the friction fit with the spine 128 of the cartridge 50. The emitters 58 and the sensors 60 of the sensor module 56 are not identified in FIG. 4, but it should be appreciated that they are coupled to the opposing inner sides 118 of the sensor housing 54 so as to be positioned adjacent or opposite the detection window 62 in the manner previously described. The spine 128 and/or the head 108 may form the handle 92 of the cartridge 50 to facilitate the cartridge 50 being inserted into and removed from the cartridge receiver 48, respectively. Additionally or alternatively, the sensor housing 54 may be coupled to the cartridge 50 prior to insertion, and thereafter itself provide a grip to manipulate the sensor-cartridge assembly for insertion into the cartridge receiver 48. The sensor housing 54 may include a dongle 130 integrated with or removably coupled to a data and power port 144 on the chassis 22 of the medical waste collection system 20 (see FIG. 6).

The cartridge receiver 48 and the cartridge 50 may include components to provide for selective locking of the cartridge 50 once removably coupled with the cartridge receiver 48. More particularly, the cartridge receiver 48 may include an actuator 120 configured to receive an input to move at least one component of the cartridge receiver 48 between two positions to selective engage a complimentary geometry of the cartridge 50. For example, the actuator 120 shown in FIG. 4A may be a tab configured to be rotated to cause geometries (not shown) internal to the cartridge receiver 48 to engage the protrusion 122 or another structure on the cartridge housing 82. In other words, the arrangement provides for “twist lock” functionality after the cartridge 50 is directed through the cartridge opening 52 and suitably positioned.

At least the detection window 62 of the spine 128 is optically clear, and FIG. 4A shows an entirety of the cartridge housing 82 being optically clear. It should be appreciated that the entirety of the cartridge housing 82 being optically clear is an optional feature, but it may simplify manufacturing by forming the cartridge housing 82 from a single material through injection molding or other suitable manufacturing technique. Further, the entirety of the cartridge housing 82 being optically clear may facilitate visualization of the color of the waste fluid being circulated through the fluid path 70 (prior to or after removal of the sensor module 56 with the cartridge 50 still inserted into the cartridge receiver 48 and the waste fluid being circulated through the fluid path 70). With the cartridge 50 is removably inserted into the cartridge receiver 48 and the sensor housing 54 coupled to the cartridge 50, the pump 80 is operated to establish the fluid path 70 from the waste volume 28 and through the inlet 72 (not shown) of the cartridge receiver 48, the outlet aperture 75 of the cartridge receiver 48, one of the bores 88 of the cartridge 50, the detection window 62, the other one of the bores 88 of the cartridge 50, the inlet aperture 73 of the cartridge receiver 48, and the outlet 74 (not shown) of the cartridge receiver 48 to be recollected in the waste volume 28. The sensor module 56 detects the optical characteristic of the waste fluid in the detection window 62 of the spine 128, and generates and transmits the optical characteristic signal to the controller 36. The controller 36 determines the volume of blood loss in the manner previously described.

Referring now to FIGS. 4B-4D, another implementation of the cartridge 50 is illustrated. The cartridge housing 82 of the cartridge 50 includes an outer diameter or outer contour complementary to an inner diameter or inner contour of the cartridge opening 52, respectively. The cartridge housing 82 includes a trunk 174, and a head 108 removably or fixedly coupled to the head 108 to define a cartridge volume. The trunk 174 of the cartridge housing 82 includes a body portion 210, a first leg 187 extending from the body portion 210, and a second leg 188 extending from the body portion 210. A void 212 is defined between the first leg 187 and the second leg 188. More particularly, each of the first leg 187 and the second leg 188 may extend proximally from the body portion 210 to define the void 212 therebetween. The void 212 is sized to receive a depth element (not shown) or another structure of the cartridge receiver 48.

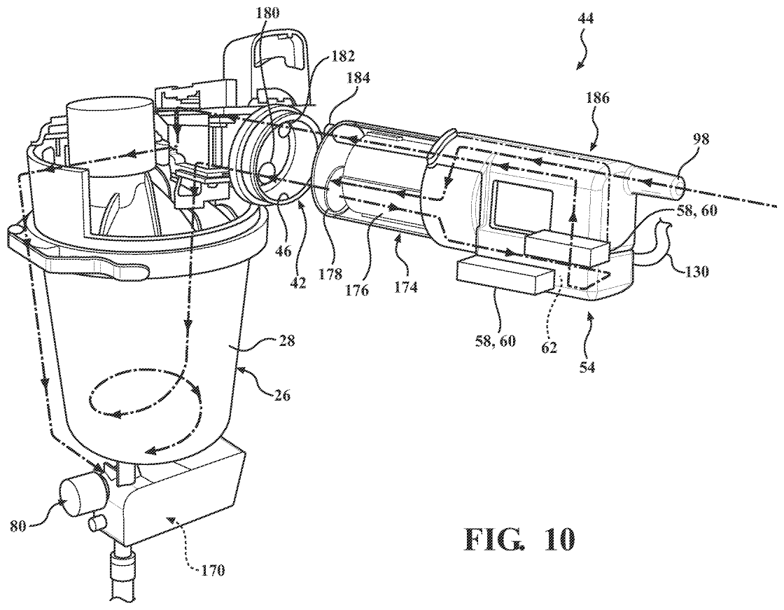

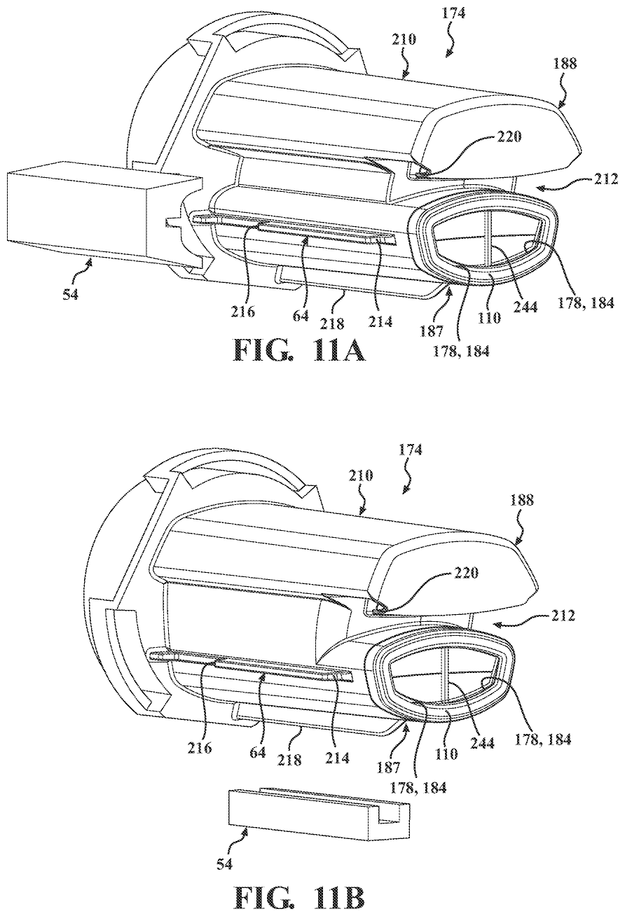

The cartridge housing 82 may include at least one of an arm 214, a lock element 216, a spine 218, and a catch 220. The illustrated implementation includes two arms 214, two lock elements 216, and two catches 220. The arm 214 may extend from the body portion 210 or the first leg 187 and include a proximally-directed surface configured to engage a sled assembly (not shown) of the cartridge receiver 48. The lock element 216 may extend from the body portion 210 and include a distally-directed surface configured to be engaged by a manifold lock assembly of the cartridge receiver 48. The spine 218 may extend from the body portion 210 or the first leg 187 include a proximally-directed surface configured to engage a sled lock assembly of the cartridge receiver 48. Lastly, the catch 220 may be disposed on the second leg 188 and include a distally-directed surface configured to be engaged by a claw (not shown) of the cartridge receiver 48. The distally-directed surface of the catch 220 may be proximal to the proximally-directed surface of the arm 214, proximal to the proximally-directed surface of the spine 218, and proximal to the distally-directed surface of the lock element 216. Further, the first leg 187 may include a rim 110 defining an outlet opening 178 and/or a recirculation opening 184 to be described. The rim 110 may be positioned distal to the distally-directed surface of the catch 220. Features of the head 108 and/or the trunk 174 may be similar to those of a manifold disclosed in commonly-owned U.S. Pat. No. 10,471,188, issued Nov. 11, 2019, the entire contents of which are hereby incorporated by reference. Further, the cartridge receiver 48 may be the same or similar to those of a manifold receiver disclosed in the aformentioned commonly-owned patent. Still further, the cartridge 50 may be devoid of an inlet fitting configured to receive a suction tube. As such, the cartridge 50 may be differentiated from that of a manifold by lacking an inlet fitting for receiving a suction tube, thereby providing a closed system in which the waste fluid is recirculated through the fluid path 70 to be recollected in the waste container 26. In implementations where the component includes an inlet fitting (see FIGS. 10 and 11), it may be considered to take the form of a recirculation manifold to be described.

With continued reference to FIG. 4B, the cartridge housing 82 defines the detection window 62 that is optically clear. For example, the detection window 62 may be disposed on the head 108, wherein the head 108 includes a ridge 128 that defines the detection window 62. The ridge 128 may be optically clear, or alternatively an entirety of the head 108 may be optically clear. The illustrated implementation shows the head 108 defining a recess 224 that is circular with the ridge 128 extending through the recess 224. The ridge 128 includes opposing outer sides 116 that are at least partially transparent. The opposing outer sides 116 may include a slight taper to facilitate a friction fit with the complementary opposing inner sides 118 of the sensor housing 54. At least the opposing inner sides 118 of the sensor housing 54 may be formed from a resilient material to facilitate the friction fit with the ridge 128 of the cartridge 50. The emitters 58 and the sensors 60 of the sensor module 56 are positioned adjacent or opposite the detection window 62 in the manner previously described. Additionally or alternatively, the sensor housing 54 may be coupled to the cartridge 50 prior to insertion, and thereafter itself provide a grip to manipulate the sensor-cartridge assembly for insertion into the cartridge receiver 48. Similarly. the cartridge 50 may include a grip 226 coupled to the head 108, the trunk 174, or another suitable structure of the cartridge 50 to facilitates insertion and removal of the cartridge 50. FIG. 4B shows the grip 226 as a band-like structure disposed about an outer circumference of the head 108 and being formed from material different than that of the head 108, such as rubber or the like, to improve handling of the cartridge 50.

As best shown in FIG. 4C, one of the first leg 187 and the second leg 188 defines the outlet opening 178, and the other one of the first leg 187 and the second leg 188 defines the recirculation opening 184. Hereinafter for convention and as represented by the arrows in FIG. 4D, the first leg 187 defines the recirculation opening 184 and the second leg 188 defines the outlet opening 178. The reverse configuration is contemplated in which the first leg 187 defines the outlet opening 178 and the second leg 188 defines the recirculation opening 184. A first seal 114 is disposed within the outlet opening 178, and a second seal 115 is disposed within the recirculation opening 184. The first seal 114 and the second seal 115 are configured to prevent egress of the waste fluid from the outlet opening 178 and the recirculation opening 184, for example, by providing face seals and radial seals with complementary fittings (not shown) of the cartridge receiver 48. The first seal 114 and the second seal 115 may be similar or different in size, shape, material selection, and the like, with FIG. 4C showing the first seal 114 as smaller than the second seal 115 but otherwise similar in construction. Further features of the first seal 114 and/or the second seal 115 may be the same or similar to those disclosed in the aformentioned commonly-owned U.S. Pat. No. 10,471,188. An opening (e.g., a slit between resilient flaps) in each of the first seal 114 and the second seal 115 are arranged on parallel axes so as to be internal to the medical waste collection system 20 to provide the closed system with lessened likelihood of exposure to the waste fluid.

In one implementation, the outlet opening 178 may be positioned below the recirculation opening 184 when the manifold 44 is oriented for insertion into the manifold opening 46 of the manifold receiver 42. The arm 214, the lock element 216, the spine 218, and the catch 220 may be positioned distal to the outlet opening 178 or the recirculation opening 184 defined in the second leg 188.

With the cartridge 50 is removably inserted into the cartridge receiver 48 and the sensor housing 54 coupled to the cartridge 50, the pump 80 is operated to establish the fluid path 70 from the waste volume 28 and through the recirculation opening 184 towards and through the detection window 62, and towards and the through the outlet opening 178 to be recollected in the waste container 26. The pump 80 may be arranged to draw a vacuum on the outlet opening 178 to draw the waste fluid through the recirculation opening 184, or alternatively the pump 80 may provide a positive pressure on the recirculation opening 184. The sensor module 56 detects the optical characteristic of the waste fluid in the detection window 62 of the ridge 128, and generates and transmits the optical characteristic signal to the controller 36. The controller 36 determines the volume of blood loss in the manner previously described.

The fluid path 70 may further includes the waste fluid being directed through the recirculation opening 184, the first leg 187 of the trunk 174, the detection window 62, the second leg 188 of the trunk 174, and the outlet opening 178. To achieve the fluid path 70, the cartridge 50 may include an inner housing 228 coupled to or otherwise engaging the trunk 174. As should be appreciated from FIG. 4D, the inner housing 228 may be at least partially disposed within the trunk 174 and include features contoured to complementary features of the trunk 174. For example, the inner housing 228 may include an inner first leg 230 disposed within the first leg 187 of the trunk 174, and an inner second leg 232 disposed within the second leg 188 of the trunk 174. The first seal 114 may be coupled to the inner second leg 232, and the second seal 115 may be coupled to the inner first leg 230. With the inner housing 228 seated within the trunk 174, the first seal 114 may be compressed between the inner second leg 232 and the second leg 188 to cover the outlet opening 178, and the second seal 115 may be compressed between the inner first leg 230 and the first leg 187 to cover the recirculation opening 184. The inner housing 228 may further include a barrier 234 separating an intake track 236 and an exhaust track 238 so as to constrain the fluid path 70 towards and away from the detection window 62, respectively. As represented by the arrows in FIG. 4D, the fluid path 70 includes the waste fluid being directed through the recirculation opening 184, the intake track 236 of the inner housing 228, the detection window 62. the exhaust track 238 of the inner housing 228, and the outlet opening 178. As previously mentioned, the reverse configuration is contemplated.

In an alternative implementation, the cartridge 50 may not include the inner housing 228, but rather the head 108 and/or the trunk 174 may be formed with internal geometries to provide the fluid path 70 previously described. Particularly in instances where the cartridge 50 is manufactured through injection molding, blow molding, and three-dimensional printing, the internal geometries necessary to provide the fluid path 70 may be readily realized without the need of a separate component.

The cartridge 50 may also include a filter element 176 disposed within the cartridge housing 53, for example, the body portion 210 of the trunk 174. FIG. 4D shows the filter clement 176 disposed within or associated with the exhaust track 238 such that the waste material being directed through the fluid path 70 is not filtered prior to encountering the detection window 62, which may affect the composition of the waste fluid being detected by the sensor module 56. However, it is contemplated that the filter element 176 or another filter element having different filtering capacity may be disposed within or associated with the intake track 236.

Referring now to FIGS. 4E-4H, alternative implementations of the cartridge 50 are shown in which the detection window 62 may be defined by structures other than the head 108. Moreover, the implementations include structures common to the previously described embodiment while providing an alternative form factor, namely a generally U-shaped cassette cartridge to be described. The cartridge housing 53 includes the body portion 210, the first leg 187 extending from the body portion 210, and the second leg 188 extending from the body portion 210. The first leg 187 and the second leg 188 may extend from the body portion 210 in the same direction to define the void 212 therebetween. One of the first leg 187 and the second leg 187 define the recirculation opening 184, and the other one of the first leg 187 and the second leg 188 define the outlet opening 178. As indicated the arrows in FIGS. 4E and 4G, the illustrated implementation includes the first leg 187 defining the outlet opening 178 and the second leg 188 defining the recirculation opening 184, but the reverse configuration is contemplated. The first seal and the second seal (not shown) may be coupled to the first leg 187 and/or the second leg 188 to cover the outlet opening 178 and the recirculation opening 184. The arm 214 extends from one of the body portion 210 and the first leg 187, the lock element 216 extends from one of the body portion 210 and the first leg 187, and the spine 218 extends from one of the body portion 210 and the first leg 187. Further, the catch 220 is disposed on the second leg 188.

Relative to the implementation of FIGS. 4B-4D in which the cartridge housing 53 has a generally cylindrical form factor, the cartridge housing of FIGS. 4E-4H include opposing flat faces 240 that are parallel and larger than opposing outer sides 242 such that the cartridge 50 is a cassette in form. The void may extend inwardly for at least half of a length of each of the first leg 187 and the second leg 188, or by any suitable distance for the cartridge housing 53 to be U-shaped. It is alternatively contemplated that protrusions (not shown) may extend from the body portion 210 in a direction opposite of the first and second legs 187, 188 such that the form factor is a generally H-shaped cassette cartridge.

FIGS. 4E and 4F show the void 212 sized and shaped to receive the sensor module 56. In such an arrangement, at least one of the first leg 187 and the second leg 188 may be considered to define the detection window 62. More particularly, the ridge 128 may extend into the void 212 from at least one of the body portion 210, the first leg 187, and the second leg 188. The ridge 128 defines the detection window 62 that is optically clear. Even more particularly, a first portion of the ridge 128 extends from the first leg 187, a second portion of the ridge 128 extends from the second leg 188, and a third portion of the ridge 128 extends from the body portion 210. With the sensor module 56 disposed within the void 212, the emitters 58 and sensors 60 of the sensor module 56 are arranged to detect the optical characteristic of the fluid within one or more portions of the detection window 62. Owing to the arrangement of the sensor module 56 along the three portions of the ridge 128, it is contemplated that several emitters and sensors may be arranged serially to take as many measurements as desired as the fluid is circulated through the cartridge 50. The potentially additional measurements may facilitate improved accuracy.

FIGS. 4G and 4H show an implementation where the spine 218 of the cartridge housing 53 defines the detection window 62. In the present implementation, the spine 218 includes the opposing outer sides 116 of the spine 218 are spaced apart from one another to define a channel in fluid communication with an interior of the body portion 210 or the first leg 187, as best shown in FIG. 4H. The opposing outer sides 116 of the spine 218 are configured to be arranged between the opposing inner sides 118 of the sensor module 56. Another variant in which the spine 218 of the cartridge housing 53 defines the detection window 62 is illustrated in FIG. 11B.

Additionally or alternatively, the arm 214 may define the detection window 62. FIG. 11A illustrates such an arrangement where the arm 214 extends from the body portion 210 and the first leg 187. Similar to FIG. 4H, the arrangement includes the arm 214 having the opposing outer sides 116 spaced apart from one another to define a channel in fluid communication with an interior of the body portion 210 or the first leg 187. It is further contemplated that other structures of the cartridge housing 53 may define the detection window 62, including but not limited to the body portion 210, the first leg 187, the second leg 188, and the catch 220.

In implementations in which the detection window 62 is associated with the arm 214, the spine 218, or other structure extending from the first leg 187, it may not be necessary for the fluid path 70 to be circulated through the second leg 188. Rather, simplified internal construction may be realized by providing both the recirculation opening 184 and the outlet opening 178 within the rim 110 of the first leg 187. With continued reference to FIGS. 11A and 11B, the first leg 187 includes the rim 110, and a barrier 244 coupled to the rim 110 to separate the recirculation opening 184 and the outlet opening 178. Geometries internal to the first leg 187 may be provided to maintain separation of the intake track and the exhaust track within the first leg 187, at least for a sufficient distance for the waste fluid to be detected by the sensor module 56. Further internal geometries may be provided to route the waste fluid from the recirculation opening 184 towards the arm 214, the spine 218, or other structure that is defining the detection window 62. It should be appreciated that the implementations illustrated in FIGS. 11A and 11B do not require the user to separately couple the sensor module 56 to the cartridge 50, thereby simplifying workflow by merely requiring intuitive insertion and removal of the cartridge 50 from the medical waste collection system 20.

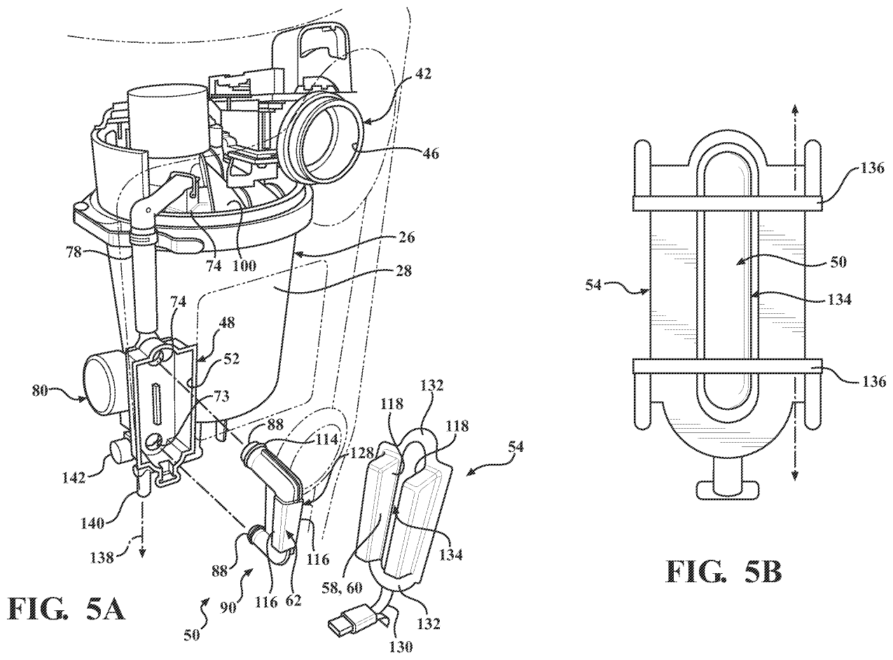

Still another implementation of the cartridge 50 is illustrated in FIGS. 5A and 5B in which the sensor module 56 is removably couplable with the cartridge 50 prior to or after insertion of the cartridge 50 into the cartridge receiver 48. A representative drawing of the waste container 26 is shown in which the cartridge receiver 48 is coupled near a bottom of the waste container 26, and the pump 80 for the fluid path 70 is coupled to the cartridge receiver 48. The cartridge receiver 48 defines the cartridge opening 52, the inlet aperture 73, and the outlet aperture 75. The cartridge receiver 48 in the implementation of FIG. 5A is sized to receive the sensor housing 54. More particularly, the cartridge opening 52 defines an inner perimeter contoured to an outer perimeter of the sensor housing 54.

The cartridge 50 may include the channel 90 or tube, but otherwise be devoid of a discrete housing supporting the cartridge 50. In other words, the tube is the cartridge 50, and the sensor housing 54 supports the tube within the cartridge receiver 48. The cartridge 50 includes the spine 128 defining the detection window 62 that is optically clear, and the entirety of the cartridge 50 may be optically clear. The shoulders 86 may extend from the spine 128 to form a U-shaped cartridge as shown. The shoulders 86 define the bores 88, and the gaskets 114 may be coupled near ends of the shoulders 86. The ends of the shoulders 86 may have an outer diameter configured to be slidably and snugly inserted into a respective one of the inlet aperture 73 and the outlet aperture 75 of the cartridge receiver 48 for the gaskets 114 to form a face seal with the inner surface 112 of the cartridge receiver 48.

The opposing outer sides 116 of the spine 128 may be friction fit with the opposing inner sides 118 of the sensor housing 54. At least the opposing inner sides 118 of the sensor housing 54 may be formed from a resilient material to facilitate the friction fit with the spine 128 of the cartridge 50, and further eliminate ambient light interference. The emitters 58 and the sensors 60 of the sensor module 56 are coupled to the opposing inner sides 118 of the sensor housing 54 so as to be positioned adjacent or opposite the detection window 62. The sensor housing 54 may include the dongle 130 integrated with or removably coupled to a data and power port 144 on the chassis 22 of the medical waste collection system 20.

The sensor housing 54 may include at least one strut 132 extending between the opposing inner sides 118 so as to permit the sensor housing 54 to define a sensor window 134. In other words, the opposing inner sides 118 are spaced apart from one another to define a slot sized to receive the cartridge 50 and permit visualization of the cartridge 50 through the sensor window 134. The sensor window 134 may be elongate and contoured to the cartridge 50. The cartridge 50 is removably inserted into the cartridge receiver 48, and the waste fluid may be visible through the sensor window 134. As shown in FIG. 5B, at least one brace or clamp 136 may be provided to secure the sensor-cartridge assembly in the cartridge receiver 48. The present arrangement provides for visualization of the waste fluid being circulated through the fluid path 70 without requiring removal of the sensor housing 54.

In arrangements where the medical waste collection system 20 includes two waste containers, certain implementations of the cartridge receiver 48 may facilitate transferring the waste fluid from a first or upper waste container to a second or lower waste container. FIG. 5A shows only the upper waste container 26 with arrow 138 indicating the flow of the waste fluid to the lower waste container. The cartridge receiver 48 defines an outport 140 in selective fluid communication between the upper waste container 26 and the lower waste container. A motor 142 may be coupled to a valve (not shown) to establish the selective fluid communication between the upper waste container 26 and the lower waste container. The implementation provides for at least a recirculation mode and a transferring mode. The recirculation mode includes operating the pump 80 to draw the waste fluid from the waste volume 28 and through the inlet 72 (not shown), the outlet aperture 75, the cartridge 50, the inlet aperture 73, the conduit 78, and the outlet 74 to be recollected in the waste volume 28. The sensor module 56 detects the optical characteristic of the waste fluid in the detection window 62, and generates and transmits the optical characteristic signal to the controller 36. The controller 36 determines the volume of blood loss in the manner previously described. The transferring mode includes operating the motor 142 to draw the waste fluid from the waste volume 28 and through the inlet 72 (not shown) and the outport 140 to be collected in the lower waste container. The transferring mode may or may not provide for the QBL analysis.

It is contemplated that the sensor module 56 may be coupled to or near the outport 140 such that the emitters 58 and the sensors 60 are arranged to detect the waste fluid being transferred from the upper waste container to the lower waste container. In such an implementation, the QBL analysis may be performed simultaneous with the emptying of the upper waste container. With the volume of the waste fluid being known from the fluid measuring system 68 in the upper waste container, the same technical advantages may be realized though the waste fluid is not recollected in the upper waste container.

Referring to FIG. 6, another implementation of the cartridge 50 is shown in which the sensor module 56 is removably couplable with the cartridge 50 prior to or after insertion of the cartridge 50 into the cartridge receiver 48. A representative drawing of the waste container 26 is shown in which the cartridge receiver 48 is coupled beneath the waste container 26, and the pump 80 is coupled to the cartridge receiver 48. The cartridge receiver 48 defines the cartridge opening 52, and the cartridge 50 is shown as already positioned within the cartridge receiver 48.

The cartridge 50 includes the spine 128 defining the detection window 62 that is optically clear, and the entirety of the cartridge 50 may be optically clear. Certain other structures common to the previous implementations of the cartridge 50, such as the bores 88, the gaskets 114, and the like, are not visible in FIG. 6, but may be considered incorporated by reference. The opposing outer sides 116 of the spine 128 may be positioned adjacent the opposing inner sides 118 of the sensor housing 54. At least the opposing inner sides 118 of the sensor housing 54 may be formed from a resilient material to facilitate the friction fit with the spine 128 of the cartridge 50, and further eliminate ambient light interference. The emitters 58 and the sensors 60 of the sensor module 56 are coupled to the opposing inner sides 118 of the sensor housing 54 so as to be positioned adjacent or opposite the detection window 62. The sensor housing 54 may include a dongle 130 configured to be removably coupled with the data and power port 144 on the chassis 22 of the medical waste collection system 20. The data and power port 144 may be a universal serial bus (USB) port.

The cartridge receiver 48 of FIG. 6 may facilitate transferring the waste fluid from the first or upper waste container 26 to the second or lower waste container. The cartridge receiver 48 defines the outport 140 in selective fluid communication between the upper waste container 26 and the lower waste container. The motor 142 may be coupled to a valve (not shown) to establish the selective fluid communication between the upper waste container 26 and the lower waste container. The implementation provides for at least the recirculation mode, the transferring mode, and a closed mode. The recirculation mode includes operating the motor 142 to position the valve, and operating the pump 80 to draw the waste fluid from the waste volume 28 and through the cartridge 50 to be recollected in the waste volume 28. The sensor module 56 detects the optical characteristic of the waste fluid in the detection window 62, and generates and transmits the optical characteristic signal to the controller 36. The controller 36 determines the volume of blood loss in the manner previously described. The transferring mode includes operating the motor 142 to position the valve to transfer the waste fluid from the waste volume 28 through the outport 140 to be collected in the lower waste container. The closed mode includes operating the motor 142 to close the valve in which the waste fluid collects within the waste volume 28 without performing QBL analysis.

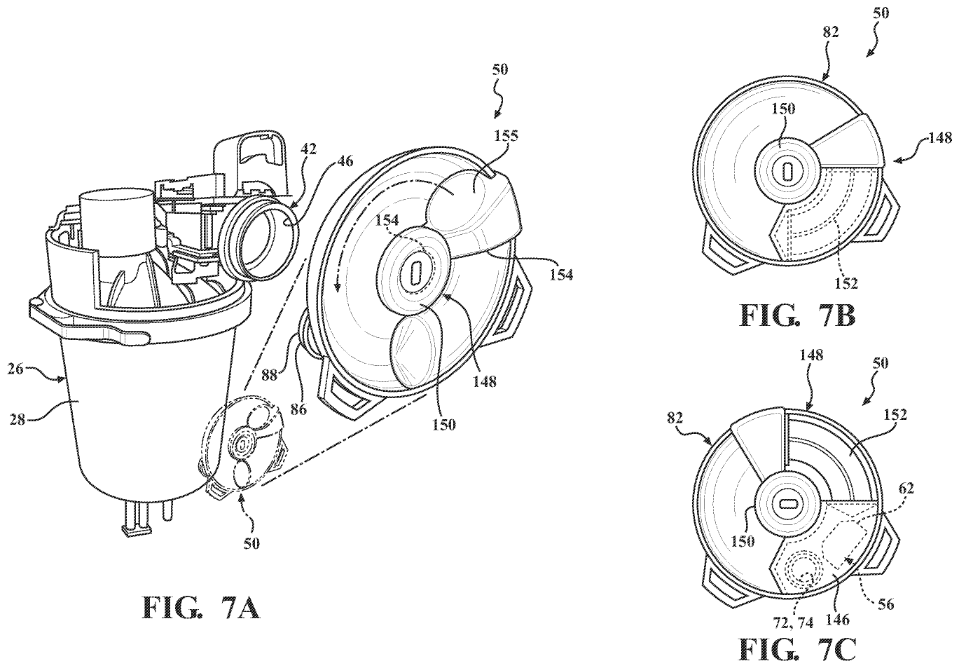

FIGS. 7A-7C show another implementation of the cartridge 50 in which the cartridge 50 defines a fluid reservoir 146 and includes means for drawing the waste fluid from waste container 26 to within the fluid reservoir 146 to facilitate the QBL analysis. The cartridge 50 includes the shoulder 86 configured to be removably coupled with the waste container 26 to establish selective fluid communication between the waste volume 28 and the fluid reservoir 146. The cartridge 50 includes an actuator 148 configured to be actuated between a closed position (FIG. 7B) in which the waste fluid is not permitted to flow between the waste volume 28 and the fluid reservoir 146, and an open position (FIG. 7C) in which the waste fluid is permitted to flow between the waste volume 28 and the fluid reservoir 146. Further, actuation of the actuator 148 may itself urge the fluid to be drawn from the waste volume 28 and into the fluid reservoir 146.

The cartridge housing 82 may be circular such that the cartridge 50 is disc-shaped for purposes of intuitive and ergonomic actuation, however, other suitable geometries are contemplated. The cartridge housing 82 defines the inlet 72, which in the present implementation also functions as the outlet 74. In other words, the waste fluid may be drawn into and discharged from the fluid reservoir 146 through the same opening. The positioning of the cartridge 50 and its function to be described may provide for the opening to be larger relative to the cartridge housing 82, which makes the opening less susceptible to clogging with passage of the waste fluid.

The actuator 148 may be pivotably coupled to the cartridge housing 82 and include a hub 150, and a plunger 152 coupled to the hub 150. The plunger 152 may be arcuate and include a curvature approximate to the radius of the cartridge housing 82. The hub 150 may be mounted near a center of the cartridge housing 82 so as to pivot about its center and move the plunger 152 in an arcuate manner. The cartridge 50 may include a motor 154 coupled to the cartridge housing 82 and configured to pivot the hub 150 relative to the cartridge housing 82 between the open and closed positions. One suitable motor is a stepper motor requiring low current. The stepper motor may be controller by the controller 36, and an input to the control panel 40 may operate the cartridge 50 to be moved between the open and closed positions. Additionally or alternatively, the hub 150 of the actuator 148 may further include at least one control surface 155 configured to receive a manual input to move the actuator 148 between the open and closed positions. With the actuator 148 in the closed position, the plunger 152 may occlude the fluid reservoir 146 and the inlet 72 opening into the fluid reservoir 146. With the actuator 148 being moved from the closed position to the open position, the plunger 152 is moved arcuately to expose the fluid reservoir 146 and the inlet 72, and the waste fluid may be drawn into the fluid reservoir 146. Other suitable actuators include a squeeze bulb, a manual syringe, a spring-loaded syringe, a motorized syringe, a pneumatic cylinder, a vacuum source, and a peristaltic pump.

At least a portion of the cartridge housing 82 is transparent to define the detection window 62. The sensor module 56 is coupled to the cartridge housing 82 such the emitters 58 and the sensors 60 are positioned adjacent or opposite the detection window 62, and further may be positioned adjacent or opposite the fluid reservoir 146. FIG. 7C schematically represents one suitable position of the sensor module 56. The actuator 148 may be actuated to draw the waste fluid under the influence of a vacuum from the waste volume 28, through the inlet 72, and into the fluid reservoir 146. The sensor module 56 detects the optical characteristic of the waste fluid in the detection window 62, and generates and transmits the optical characteristic signal to the controller 36. The controller 36 determines the volume of blood loss in the manner previously described. The actuator 148 may be further actuated for the plunger 152 to urge the waste fluid from the fluid reservoir 146, through the outlet 74, to be recollected in the waste volume 28. Owing to the relative dimensions of the plunger 152 and the fluid reservoir 146, the plunger 152 may itself or include a sealing member (not shown) to slidably or frictionally remove or clean residual amounts of the waste fluid from the detection window 62. The self-cleaning may extend an operational life of the cartridge 50 by minimizing fouling of the detection window 62, or alternatively it may provide for the functionality of the cartridge 50 be integrated onto the waste container 26 as a capital component. It should be understood that the step of actuating the actuator 148 between the open and closed positions may be performed quickly and repeatedly as often as desired or indicated to obtain an updated QBL analysis. Moreover, the time to perform the QBL analysis (e.g., time with which the sensors 60 detect the optical characteristic, and controller 36 processes the signals) may be near-instantaneous.

FIGS. 8A-8C show another implementation in which there is the fluid reservoir 146 to facilitate the QBL analysis. The medical waste collection system 20 may include the manifold receivers 42 in fluid communication with the waste containers 26 (not identified), and further include a tank 156 that defines the fluid reservoir 146. The tank 156 may be integrated with or removably coupled to the chassis 22, or may be removably coupled to a cradle 158 that is mounted to the chassis 22. The cradle 158 may include retention features (not shown) configured to releasably engage complementary retention features of the tank 156. The retention features may be actuated between engaged and disengaged positions. The actuation may be provided through a button 160 on the cradle 158, or an input on the control panel 40 in which the controller 36 electronically moves the structure to the disengaged position to permit removal of the tank 156 from the cradle 158 or the chassis 22.

The tank 156 may include a base 162, and a lid 164 coupled to the base 162 to define the fluid reservoir 146. The base 162 or the lid 164 may be at least partially clear to define the detection window 62. FIG. 8C shows the base 162 including the spine 128 extending from a sidewall of the tank 156. The spine 128 being integrated on the tank 156 may eliminate clogging by eliminating the waste fluid passing through openings of relatively smaller diameters. FIG. 8C further shows the opposing outer sides 116 of the spine 128 being coupled with the opposing inner sides 118 of the sensor housing 54. At least the opposing inner sides 118 of the sensor housing 54 may be formed from a resilient material to facilitate a friction fit with the spine 128 of the cartridge 50, and further eliminate ambient light interference. Alternatively, the sensor housing 54 may be slidably inserted into a slot or pocket to be removably coupled to the base 162 in a position adjacent the detection window 62. The emitters 58 and the sensors 60 of the sensor module 56 are coupled to the opposing inner sides 118 of the sensor housing 54 so as to be positioned adjacent or opposite the detection window 62. The sensor housing 54 may include the dongle 130 integrated with or removably coupled to a data and power port 144 on the chassis 22 of the medical waste collection system 20.

The base 162 or the lid 164 may include at least one inlet fitting 166, and at least one outlet fitting 168. The inlet fitting 166 and the outlet fitting 168 are configured to be removably coupled to a suction tube, with the resulting inflows and outflow schematically represented by arrows in FIG. 8B. In one example, the suction tube coupled to the outlet fitting 168 may further be coupled to the inlet fitting 98 of the manifold 44 removably inserted into the manifold receiver 42 (see FIG. 1). The suction tube coupled to the inlet fitting 166 becomes a distal suction tube to which a surgical instrument may be coupled. The vacuum provided by the vacuum source 30 is configured to draw the waste fluid through the distal suction tube to be collected in the fluid reservoir 146. At a certain fluid level, the waste fluid is disposed within the detection window 62, and the sensor module 56 detects the optical characteristic of the waste fluid in the detection window 62, and generates and transmits the optical characteristic signal to the controller 36. The controller 36 determines the volume of blood loss in the manner previously described.

A valve 170 may be arranged in fluid communication with the fluid reservoir 146 to establish the selective fluid communication between the fluid reservoir 146 and the waste container 26 disposed on the chassis 22. FIG. 8B schematically represents the valve 170 positioned below the base 162 of the tank 156, for example, coupled to the cradle 158. The valve 170 may include at least the transferring mode and the closed mode. The transferring mode includes positioning the valve 170 to transfer the waste fluid from the fluid reservoir 146 through the outport 140 to be collected in the waste container 26. The waste fluid may be drawn from the fluid reservoir 146 and into the waste volume 28 under influence of the vacuum from the vacuum source 30. The closed mode includes positioning the valve 170 such that the waste fluid collects within the fluid reservoir 146 for the QBL analysis to be performed. It should be appreciated that the QBL analysis may be performed in the transferring mode in a manner previously described. For example, the emitters 58 and the sensors 60 are arranged to detect the waste fluid being transferred from the fluid reservoir 146 to the waste container 26. In such an implementation, the QBL analysis may be performed simultaneous with the emptying of the fluid reservoir 146.

In certain implementations, the entirety of the base 162 or the lid 164 may be optically clear may facilitate visualization of the color of the waste fluid being circulated through the fluid path 70, as appreciated from FIG. 8A. It is contemplated that a removable or openable cover may be coupled to or integrated with the tank 156 so as to limit visibility as desired, for example, during labor and delivery or other surgical procedures in which non-medical persons may be present. The tank 156 may include the agitator 126 configured to facilitate homogenization of the waste fluid within the waste volume 28. The agitator 126 includes the impeller within the fluid reservoir 146 and rotated by the magnetic drive to mix the waste fluid in the fluid reservoir 146.

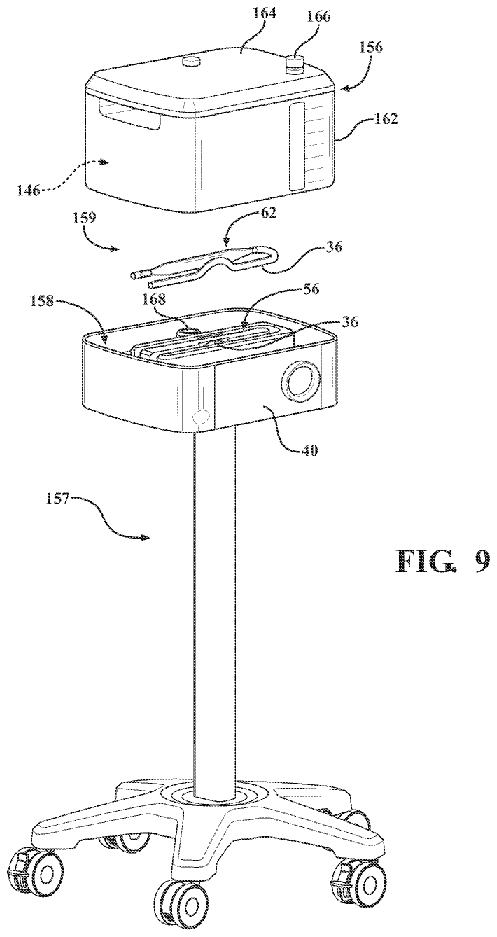

In another example, the suction tube coupled to the outlet fitting 168 of the tank 156 may further be coupled to a separate vacuum source, such as a vacuum system integrated with the operating theatre. In such an arrangement, the tank 156 may be used as a standalone unit. FIG. 9 shows such an arrangement in which the tank 156 is supported atop a stand 157. The stand 157 includes the cradle 158 within which the tank 156 may be removably supported. The stand 157 may also include the control panel 40. The control panel 40 may include a display, such as a touchscreen display, and one or more inputs, such as a knob. The stand 157 may also support the pump 80, for example, a peristaltic pump disposed within geometries defined by the cradle 158. The sensor housing 54 may also be disposed within the geometries defined by the cradle 158.

The geometries are sized and shaped to receive a detection window assembly 159, which includes tubing 161 and an optically clear component defining the detection window 62. The tubing 161 includes opposing ends each coupled to fittings (not shown) on an underside of the base 162 of the tank 156. The lid 164 of the tank 156 includes the inlet fitting 166, whereas the outlet fitting 168 may be disposed within the cradle 158. The inlet fitting 166 is couplable to a suction tube configured to draw fluids from the patient, and the outlet fitting 168 is couplable to another suction tube an arranged in fluid communication with an external vacuum source. The external vacuum source may the vacuum source 30 previously described, or a facility-integrated vacuum system. The vacuum source 30 draws the fluids into the fluid reservoir 146 of the tank

With the tank 156 supported on the cradle 158, the tubing 161 is in operable communication with the pump 80. Upon an input to the control panel 40 or as otherwise indicated, the pump 80 causes the waste fluids to pass through the tubing 161 and the detection window 62. The sensor module 56 operates in manners previously described to obtain the data indicated for the QBL analysis. Once it is desired to empty the fluid reservoir 146, the suction tube coupled to the inlet fitting 166 may be arranged in fluid communication with the vacuum source 30, for example, via the manifold 44 of the medical waste collection system of FIG. 1. The waste fluids may be drawn under suction from the tank 156 and into the waste container 26 of the medical waste collection system 20. Additionally or alternatively, the tank 156 may be removed from the cradle 158 and disposed of according to acceptable disposal practices for the medical facility.

Returning to FIGS. 1 and 3, and further with reference to FIGS. 10-12, the QBL analysis may be performed by coupling the sensor module 56 to the manifold 44, and recirculating the waste fluid from the waste volume 28 and through the manifold 44. The arrangement may be an alternative or in addition to the implementations in which the cartridge 50 is removably coupled to the cartridge receiver 48. FIG. 10 shows a representative drawing of the waste container 26 in which the manifold receiver 42 is coupled to the cap 100 of the waste container 26, and a trunk 174 of the manifold 44 is positioned near the manifold opening 46. The dashed lines illustrate the circulation and recirculation of the waste fluid through the manifold 44. The suction tube is coupled to the inlet fitting 98, and the vacuum from the vacuum source 30 draws the waste fluid from the surgical field through the suction tube and into the manifold 44. The waste fluid is drawn through a bypass flow path in which it is not directed through the detection window 62. The bypass flow path includes the waste fluid being directed through a filter element 176 disposed within the trunk 174, and an outlet opening 178. Once exiting the manifold 44 through the outlet opening 178, a container inflow path includes the waste fluid being drawn through an intake 180 of the manifold receiver 42 and into the waste container 26. The fluid measurement system 68 is configured to generate the level signal representative of a fluid level of the waste fluid collected within the waste container 26, and transmit the waste level signal to the controller 36. Once it is indicated to perform the QBL analysis, the pump 80 may be operated to recirculate the waste fluid in a container outflow path from the waste volume 28 through a discharge 182 of the manifold receiver 42 and into a recirculation opening 184 defined by the trunk 174 of the manifold 44. FIG. 10 shows the outlet opening 178 and the recirculation opening 184 being defined a proximal base of the trunk 174.

The waste fluid is directed towards the sensor module 56 coupled to a head 186 of the manifold 44, and further directed through the detection window (approximated in FIG. 10) positioned between the emitters 58 and the sensors 60 of the sensor module 56. The waste fluid may bypass the filter clement 176 as it is recirculated towards the sensor module 56. The sensor module 56 detects the optical characteristic of the waste fluid in the detection window 62, and generates and transmits the optical characteristic signal to the controller 36. The controller 36 determines the volume of blood loss in the manner previously described. The flow path may be rejoined with the bypass path at a position proximal to the sensor module 56 to again be directed through the filter element 176 and the outlet opening 178. The recirculation may be continuous, intermittent, or controlled as desired on the control panel 40. Further, the manifold 44 may be disposable after a single use, thereby obviating fouling concerns and further limiting exposure to the waste fluid through known protective features disclosed in commonly-owned U.S. Pat. No. 7,615,037, issued Nov. 10, 2009, the entire contents of which are hereby incorporated by reference. The manifold 44 may include an RFID tag including memory storing data indicative of compatibility of the manifold 44, and/or instructions for operating the medical waste collection system 20 in a particular manner with the manifold 44 for facilitating the QBL analysis.

FIG. 3 illustrates another implementation of the manifold 44 in which the outlet opening 178 is defined in the first leg 187 of the trunk 174, and the recirculation opening 184 is defined in a second leg 188 of the trunk 174. Certain features of the manifold 44 of FIG. 3 are further described in commonly-owned U.S. Pat. No. 10,471,188, issued Nov. 11, 2019, the entire contents of which are hereby incorporated by reference, in particular arms, lock elements, a spine, and catches. The outlet opening 178 may be positioned below the recirculation opening 184 when the manifold 44 is oriented for insertion into the manifold opening 46 of the manifold receiver 42. The first leg 187 and the second leg 188 may be spaced apart from one another to at least partially define the void sized to receive a depth element (not identified) of an inlet mechanism 190. The arms, the lock elements, the spine, and the catches may be positioned distal to the recirculation opening 184 defined in the second leg 188.

The manifold 44 is configured to be inserted into and removed from the manifold receiver 42 in a proximal direction and a distal direction, respectively. The inlet mechanism 190 is configured to move in the proximal and distal directions opposite of a direction of movement of the manifold 44 to prevent fluid communication between the manifold 44 and the vacuum source 30 until the manifold 44 is in a fully inserted operative position. The inlet mechanism 190 may define a first pathway 192 and a second pathway 194 configured to establish the fluid communication between the manifold 44 and the waste container 26. More particularly, the first pathway 192 is configured to be arranged in fluid communication with the outlet opening 178 of the manifold 44, and the second pathway 194 is configured to be arranged in fluid communication with the recirculation opening 184 of the manifold 44. The first pathway 192 may define a portion of the container inflow path, and the second pathway 194 may define a portion of the container outflow path. More particularly, the vacuum from the vacuum source 30 through the first pathway 192 draws the waste fluid from the surgical field through the suction tube and into the manifold 44. The waste fluid is drawn through the first leg 187, the outlet opening 178, and the first pathway 192 to be collected in the waste volume 28. The fluid measurement system 68 is configured to generate and transmit the waste level signal to the controller 36. Once it is indicated to perform the QBL analysis, another pump (not identified) may be operated to recirculate the waste fluid from the waste volume 28 through the second pathway 194 and into recirculation opening 184 defined by the second leg 188 of the trunk 174 of the manifold 44.

The waste fluid is directed towards the sensor module 56 coupled to the head 186 of the manifold 44, and further directed through the detection window 62. The sensor module 56 detects the optical characteristic of the waste fluid in the detection window 62, and generates and transmits the optical characteristic signal to the controller 36. The controller 36 determines the volume of blood loss in the manner previously described. The flow path may be rejoined with the bypass path and be again directed through the filter element 176 and the outlet opening 178 to be collected in the waste container 26.

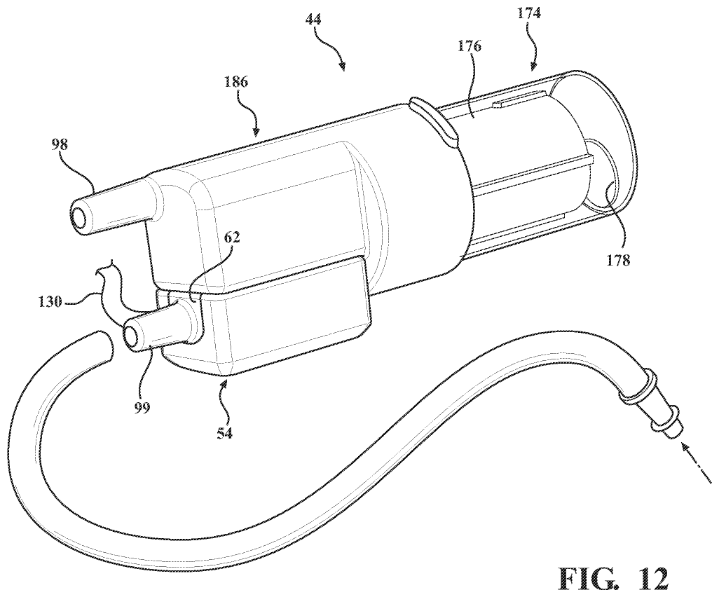

Referring to FIG. 12, another implementation of the manifold 44 is shown in which a second inlet fitting 99 is configured to be coupled with a suction tube that itself is coupled to discharge port (not shown) of the waste container 26. Whereas FIG. 10 shows the container outflow path being internal to or integrated with the chassis 22, the implementation of FIG. 12 may be considered recirculating flow path that is external to the chassis 22. The arrangement may simplify construction of the medical waste collection system 20 by eliminating the need for certain pathways within the movable components of the manifold receiver 42 such as the inlet mechanism 190. FIG. 12 shows the second inlet fitting 99 being disposed on the head 186 and positioned below the first inlet fitting 98. The second inlet fitting 99 is configured to receive the waste fluid being recirculated by the pump 80. The sensor module 56 is positioned proximal to the second inlet fitting 99 and on opposing sides of the detection window 62.

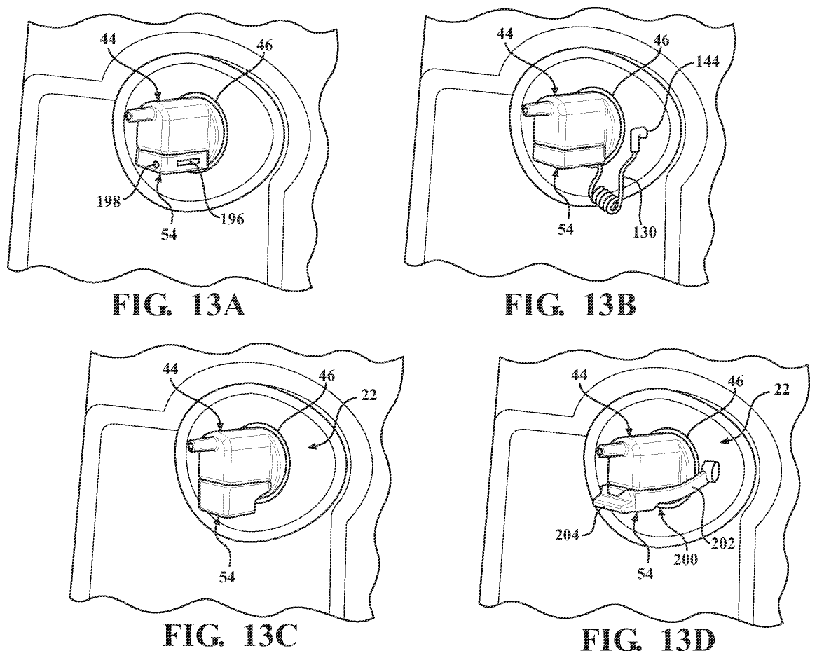

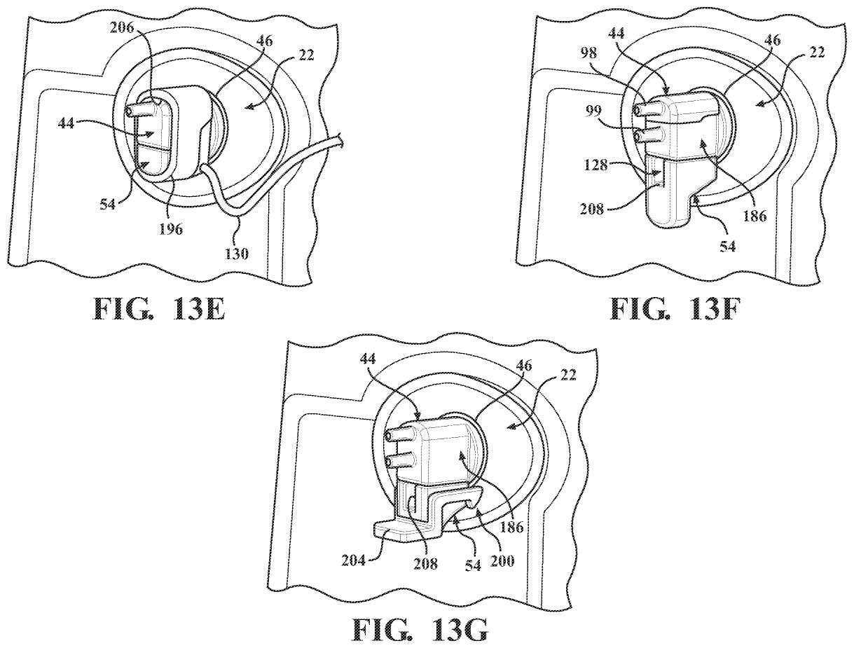

For implementations in which the manifold 44 is configured to be removably coupled with the sensor module 56, FIGS. 13A-13G show various arrangements in which this may be intuitively and ergonomically accomplished. Starting with FIG. 13A, the sensor module 56 may be battery-powered and either disposable or reusable. The sensor module 56 includes the wireless communication module configured to transmit data to a receiver on the chassis 22. One suitable communications protocol is Bluetooth low energy to extend operational life of the battery. The battery is configured to power at least the emitters 58, the sensors 60, and the wireless communication module. The sensor housing 54 may include retention features 196 configured to engage complementary retention features of the manifold 44. An indicator 198 may be coupled to the sensor housing 54 and provide visual or audible alerts related to the operation of the sensor module 56. Such alerts may be for low battery, active QBL analysis, excessive blood loss, or the like. FIG. 13B may be similar in certain respects to the arrangement of FIG. 13A, and further include the dongle 130 configured to be coupled to the data and power port 144 of the chassis or another console. The arrangement of FIG. 13B may not require the battery and/or the wireless communications module. FIG. 13C may be similar in certain respects to the arrangement of FIG. 13A, removably coupled with the chassis 22 as opposed to the manifold 44. Suitable mechanisms may be included on the manifold receiver 42 to releasably secure the sensor housing 54 in a positioned adjacent the manifold 44. FIG. 13D shows an arrangement in which the sensor housing 54 includes a stirrup 200 pivotably coupled to the chassis 22. The stirrup 200 includes legs 202 configured to be positioned on opposing sides of the manifold 44. Power and data connections may be facilities through the legs 202 being coupled to the chassis 22. The stirrup 200 may include an actuator 204 configured to receive an input to pivot the stirrup 200 between an engaged position in which the sensor module 56 is positioned on opposing sides of the detection window 62 (not identified), and a release position where the manifold 44 may be removed from the manifold receiver 42. The stirrup 200 may include mechanisms, such as detents, biasing members, and the like, to facilitate the movement between the engaged and release positions. FIG. 13E shows an arrangement in which the sensor housing 54 defines an aperture 206 shaped to a periphery of the manifold 44. The sensor housing 54 may include the retention features 196 configured to engage complementary retention features of the manifold 44. The sensor module 56 may be coupled to the sensor housing 54 such that the sensor module 56 is positioned adjacent a lower aspect of the manifold 44. The sensor module 56 may include the dongle 130. FIGS. 13F and 13G show arrangements where the manifold 44 includes the spine 128 extending downwardly from the head 186 of the manifold 44, and the sensor housing 54 defining a slot 208 configured to slidably receive the spine 128. FIG. 13F shows the sensor housing 54 integrated with the chassis 22, whereas FIG. 13G shows the stirrup 200 supporting the sensor housing 54. The stirrup 200 is coupled to the chassis 22 with the actuator 204 configured to receive an input to pivot the stirrup 200 relative to the chassis 22 between the engaged position in which the sensor housing 54 is supported adjacent the manifold 44, and the release position where the sensor housing 54 may be removed. FIG. 13F shows the first inlet fitting 98 providing a flow path bypassing the sensor module 56, and the second inlet fitting 99 providing another flow path through the detection window 62 of the spine 128, whereas FIG. 13G shows two of the second inlet fittings 99 configured to simultaneously provide for the QBL analysis through two suction tubes.

Certain implementations may be described with reference to the following exemplary clauses.

Clause 1—A medical waste collection system for performing quantitative blood loss analysis of waste fluid, the medical waste collection system including: a chassis; a vacuum source supported on the chassis; a waste container supported on the chassis and defining a waste volume; a manifold receiver supported on the chassis and configured to removably receive a manifold, wherein the vacuum source is configured to drawn a vacuum through the manifold receiver for the waste fluid to be collected in the waste volume; a cartridge receiver supported on the chassis and configured to removably receive a cartridge to form a fluid path through which the waste fluid may be circulated from the waste volume to be recollected in the waste volume; and a sensor module including emitters and optical detectors arranged to be adjacent or opposite a detection window of the cartridge disposed within the cartridge receiver.

Clause 2—The medical waste collection system of clause 1, wherein the sensor module is coupled to the cartridge receiver.

Clause 3—The medical waste collection system of clause 2, wherein the sensor module is configured to be removably coupled to the cartridge receiver.

Clause 4—The medical waste collection system of clause 3, wherein the sensor module is configured to be removably coupled to the cartridge.

Clause 5—The medical waste collection system of clause 4, wherein a sensor housing to which the sensor module is coupled is at least partially formed from resilient material so as to be removably coupled to the cartridge with a friction fit.

Clause 6—A medical waste collection system for performing quantitative blood loss analysis of waste fluid, the medical waste collection system including: a waste container defining a waste volume configured to collect the waste fluid; a fluid path defining an inlet and an outlet in fluid communication with the waste container; a pump in fluid communication with the waste container and configured to circulate the waste fluid from the waste container through the fluid path to be recollected within the waste container; and a sensor module including a light emitter and an optical detector, the sensor module arranged to detect an optical characteristic indicative of a blood concentration of the waste fluid being circulated through the fluid path.

Clause 7—The medical waste collection system of clause 6, further including a vacuum source in fluid communication with the waste container and configured to draw the waste fluid under the influence of suction to be collected within the waste container.

Clause 8—The medical waste collection system of clause 6 or 7, wherein the fluid path further includes a conduit coupled to an exterior of the waste container to define the inlet and the outlet, wherein the sensor module and the pump are coupled to the conduit.

Clause 9—A medical waste collection system for performing quantitative blood loss analysis of waste fluid, the medical waste collection system including: a waste container defining a waste volume configured to collect the waste fluid; a vacuum source in fluid communication with the waste container and configured to draw waste fluid into the waste container under the influence of suction; a pump in fluid communication with the waste container; and a sensor module including a light emitter and an optical detector, the sensor module arranged to detect an optical characteristic indicative of a blood concentration of the waste fluid during circulation of the waste fluid under influence of positive or negative pressure from the pump.

Clause 10—A medical waste collection system for performing quantitative blood loss analysis of waste fluid, the system including: a waste container defining a waste volume configured to collect the waste fluid; a fluid path defining an inlet and an outlet in fluid communication with the waste container, wherein the inlet is located below the outlet; and a sensor module including a light emitter and an optical detector, the sensor module arranged to detect an optical characteristic indicative of blood concentration of the waste fluid being circulated through the fluid path.

Clause 11—The medical waste collection system of clause 10, wherein the waste container is configured to be prefilled with the waste fluid or another fluid to a fluid level above the inlet of the fluid path prior to operation of the sensor module.

Clause 12—The medical waste collection system of clause 10 or 11, further including a pump in fluid communication with the waste container and configured to pump the waste fluid through the fluid path against gravity.

Clause 13—The medical waste collection system of clause 12, further including a vacuum source separate from the pump and in fluid communication with the waste container and configured to draw waste fluid into the waste container under the influence of suction.

Clause 14—The medical waste collection system of any one of clauses 6-13, further including: a level sensor coupled to the waste container and configured to measure a level of the waste fluid; and a controller in electronic communication with the sensor module and the level sensor, the controller configured to quantify a volume of blood loss of the waste fluid based on the blood concentration and a waste volume determined from the level of the waste fluid.

Clause 15—A medical waste collection system for performing quantitative blood loss analysis of waste fluid, the system including: a waste container defining a waste volume configured to collect the waste fluid; a level sensor coupled to the waste container and configured to measure a level of the waste fluid within the waste volume and generate a level signal; a sensor module including a light emitter and an optical detector, the sensor module arranged to detect an optical characteristic indicative of a blood concentration of the waste fluid and generate an optical characteristic signal; and a controller in electronic communication with the level sensor and the sensor module, the controller configured to: determine a waste volume of the waste fluid based on the level signal; determine a blood concentration of the waste fluid based on the optical characteristic signal; and determine a volume of blood loss based on the waste volume and the blood concentration.

Clause 16—The medical waste collection system of any one of clauses 1-15, further including an agitator disposed within the waste container and configured to facilitate homogenization of the waste fluid within the waste container.

Clause 17—The medical waste collection system of any one of clauses 6-16, further including: a cartridge receiver coupled to the waste container with the sensor module coupled to the cartridge receiver; and a cartridge configured to be removably coupled with the cartridge receiver.

Clause 18—The medical waste collection system of clause 17, wherein the cartridge further includes a tube that is optically clear so as to define a detection window configured to be arranged between the light emitter and the optical detector with the cartridge being removably coupled to the cartridge receiver.

Clause 19—A medical waste collection system for performing quantitative blood loss analysis of waste fluid, the system including: a waste container defining a waste volume configured to collect the waste fluid; a pump in fluid communication with the waste container; a sensor module including a light emitter and an optical detector; and a cartridge defining a passageway configured to be removably coupled with the sensor module to define a fluid path, and a detection window that is optically clear through which the sensor module is configured to detect an optical characteristic indicative of a blood concentration of the waste fluid during circulation of the waste fluid under influence of positive or negative pressure from the pump.

Clause 20—The medical waste collection system of clause 19, wherein the sensor module includes a cartridge receiver defining a void, and wherein the cartridge further includes a cartridge housing sized to be at least partially disposed within the void.

Clause 21—The medical waste collection system of clause 20, wherein the cartridge receiver is coupled to the waste container.

Clause 22—The medical waste collection system of any one of clauses 19-21, wherein the cartridge housing further includes retention features configured to releasably engage complementary retention features on the cartridge receiver.

Clause 23—The medical waste collection system of any one of clauses 19-22, wherein the sensor module includes a dongle, and a sensor head coupled to the dongle and including the light emitter and the optical detector, wherein the sensor head is configured to be removably coupled with the cartridge.

Clause 24—The medical waste collection system of any one of clauses 19-22, wherein the sensor module is integrated with the waste container.

Clause 25—The medical waste collection system of any one of clauses 19-24, wherein the cartridge includes a radiofrequency identification (RFID) tag including memory storing data indicative of compatibility of the cartridge with the system.

Clause 26—A medical waste collection system for performing quantitative blood loss analysis of waste fluid, the system including: a chassis; a waste container on the chassis and defining a waste volume configured to collect the waste fluid; a vacuum source supported on the chassis; a tank coupled to the chassis and defining a fluid reservoir and defining a detection window that is optically clear; and a sensor housing configured to be coupled to the tank to position a sensor module on opposite sides of the detection window to detect an optical characteristic indicative of a blood concentration of the waste fluid.

Clause 27—The medical waste collection system of clause 26, wherein the tank is configured to be arranged in fluid communication with the vacuum source.

Clause 28—The medical waste collection system of clause 27, further including a manifold receiver supported on the chassis and configured to removably receive a manifold, wherein the tank is further configured to be arranged in in fluid communication with the vacuum source through the manifold.

Clause 29—The medical waste collection system of clause 26, wherein the tank is configured to be arranged in fluid communication with a remove vacuum source within the medical facility.

Clause 30—The medical waste collection system of any one of clauses 26-29, wherein the tank includes a base having sidewalls, wherein the spine defining the detection window extends outwardly from one of the sidewalls.

Clause 31—The medical waste collection system of any one of clauses 26-30, further including a valve in selective fluid communication of an outport of the tank, wherein the valve is configured to be actuated to permit the vacuum source to draw the waste fluid from the fluid reservoir and into the waste container.

Clause 32—The medical waste collection system of any one of clauses 26-31, further including a cradle mounted on the chassis, wherein the tank is removably coupled to the cradle.

Clause 33—A medical waste collection system for performing quantitative blood loss analysis of waste fluid, the system including: a chassis; a waste container supported on the chassis and defining a waste volume; a manifold receiver supported on the chassis and configured to removably receive a manifold; a vacuum source supported on the chassis and in fluid communication with the waste volume, wherein the vacuum source is configured to draw the waste fluid through the manifold to be collected in the waste volume; a cartridge receiver separate from the manifold receiver and configured to removably receive a cartridge forming a fluid path in fluid communication with the waste volume.

Clause 34—The medical waste collection system of clause 33, further including a pump separate from the vacuum source and configured to circulate the waste fluid from the waste volume through the cartridge to be recollected in the waste volume.

Clause 35—The medical waste collection system of clause 33 or 34, wherein the cartridge receiver includes a sensor housing, and a sensor module coupled to the sensor housing.

Clause 36—A manifold for performing quantitative blood loss analysis of waste fluid with a medical waste collection system including a waste container, a vacuum source, a manifold receiver, and a sensor module, the manifold including: a trunk configured to be removably inserted into the manifold receiver, the trunk defining an outlet opening; a head coupled to the trunk to define a manifold volume, the head including an inlet fitting configured to removably receive a suction tube, and defining a detection window configured to be arranged between emitters and sensors of the sensor module; and a filter element disposed within the manifold volume, wherein one of the trunk and the head further defines a recirculation opening. wherein the manifold defines an initial flow path from the inlet fitting through the filter element and the outlet opening, and wherein the manifold defines a recirculation flow path from the recirculation opening through the detection window and the opening outlet opening.

Clause 37—The manifold of clause 36, wherein the recirculation opening is defined by the trunk.

Clause 38—The manifold of clause 37, wherein the trunk further includes a first leg defining the outlet opening, and a second leg defining the recirculation opening, wherein the first leg and the second leg are separated by a void.

Clause 39—The manifold of clause 36, wherein the recirculation opening is defined by the head.

Clause 40—The manifold of clause 39, wherein the head further includes a second inlet fitting that define the recirculation opening.

Clause 41—The manifold of any one of clauses 36-40, wherein the waste fluid in the initial flow pass does not pass through the detection window.

Clause 42—The manifold of any one of clauses 36-41, wherein the waste fluid in the recirculation path does not pass through the filter element.

Clause 43—A method of performing quantitative blood loss analysis of waste fluid with a medical waste collection system including a waste container, a vacuum source, a manifold receiver, a cartridge receiver, and a sensor module, the method including: removably coupling a manifold with the manifold receiver; coupling a suction tube to the manifold; removably coupling a cartridge the cartridge receiver to form a fluid path through the cartridge and the cartridge receiver, wherein the cartridge defines a detection window; operating the vacuum source to draw the waste fluid from a surgical site through the suction tube and the manifold to be collected within the waste container; operating the vacuum source or a pump to circulate the waste fluid from the waste container and through the fluid path to be recollected within the waste container; and detecting with the sensor module an optical characteristic of the waste fluid in the fluid path through the detection window of the cartridge.

Clause 44—The method of clause 43, wherein the medical waste collection system includes the pump, the method including operating the pump to circulate the waste fluid from the waste container and through the fluid path to be recollected within the waste container.

Clause 45—The method of clause 43 or 44, further including coupling the sensor module with the cartridge to position emitters and optical detectors of the sensor module on opposing sides of the detection window.

Clause 46—The method of clause 45, further including coupling the sensor module with the cartridge prior to the step of coupling the cartridge with the cartridge receiver.

Clause 47—The method of any one of clauses 43-46, actuating an actuator to selectively lock to one another the cartridge and the cartridge receiver.

Clause 48—A method of performing quantitative blood loss analysis of waste fluid with a medical waste collection system including a waste container, a vacuum source, a manifold receiver, a sensor module, and a controller in communication with the sensor module, the method including: collecting the waste fluid in the waste container under a vacuum generated by the vacuum source; determining with the controller a volume of the waste fluid in the waste container; circulating with the vacuum source or another pump the waste fluid from the waste container and through a fluid path to be recollected within the waste container; and detecting with the sensor module an optical characteristic of the waste fluid in the fluid path; determining with the controller a blood concentration of the waste fluid; and determining with the controller a volume of blood loss based on the volume of the waste fluid and the blood concentration of the waste fluid.

Clause 49—The method of clause 48, wherein the medical waste collection system further includes a cartridge receiver, the method further including: removably receiving a cartridge in the cartridge receiver; circulating the waste fluid through the cartridge; and detecting the optical characteristic of the waste fluid in the cartridge.

Clause 50—The method of clause 49, further including removably coupling the sensor module with the cartridge to position emitters and optical detectors of the sensor module on opposing sides of the cartridge.

Clause 51—The method of clause 48, further including: removably receiving a manifold in the manifold receiver, wherein the waste fluid collected in the waste container under the vacuum is drawn through the manifold; circulating the waste fluid through the manifold to be recollected din the waste container; and detecting the optical characteristic of the waste fluid in the manifold.

Clause 52—The method of clause 51, further including removably coupling the sensor module with the manifold to position emitters and optical detectors of the sensor module on opposing sides of the manifold.

Clause 53—A method of performing quantitative blood loss analysis of waste fluid with a medical waste collection system including a waste container, a vacuum source, a manifold receiver, and a sensor module, the method including: removably coupling a manifold with the manifold receiver, wherein the manifold defines a detection window; coupling a suction tube to the manifold; operating the vacuum source to draw the waste fluid from a surgical site through the suction tube and the manifold to be collected within the waste container; operating the vacuum source or a pump to recirculate the waste fluid from the waste container and again through the manifold be recollected within the waste container; and detecting with the sensor module an optical characteristic of the waste fluid through the detection window of the manifold.

Clause 54—The method of clause 53, wherein the suction tube is a first suction tube coupled to a first inlet fitting of the manifold, the method further including coupling a second suction tube to a second inlet fitting on the manifold, wherein drawing of the waste fluid is through the first inlet fitting and the recirculating of the waste fluid is through the second inlet fitting.

Clause 55—The method of clause 53, wherein the step of removably coupling a manifold with the manifold receiver further includes inserting the manifold in a proximal direction to establish fluid communication between a recirculation opening of the manifold with a pathway of the manifold receiver.

Clause 56—A method of determining a volume of blood collected with a system including a waste container, a fluid path, a sensor module including a light emitter and an optical detector, a pump in fluid communication with the waste container, a level sensor, and a controller in communication with the pump, the sensor module, and the level sensor, the method including: receiving waste fluid within the waste container; detecting with the level sensor a waste level of the waste fluid within the waste container, and transmitting a level signal to the controller; determining with the controller a volume of waste within the waste container based on the level signal and a known volume of the waste container; circulating with the pump the waste fluid through the fluid path to be recollected in the waste container; detecting with the sensor module an optical characteristic of the waste fluid with the optical characteristic being indicative of a blood concentration within the waste fluid, and transmitting a corresponding concentrations signal to the controller; and determining with the controller the volume of blood collected based on the volume of waste and the blood concentration.

Clause 57—The method of clause 56, wherein the system includes a display, the method further including displaying with the display the volume of blood collected.

Clause 58—The method of clause 56 or 57, further including providing an audible or visual alert if the volume of blood collected exceeds a predetermined value.

Clause 59—The method of any one of clauses 56-58, further including prefilling the waste container with the waste fluid or another fluid to a fluid level above an inlet of the fluid path prior to the step of circulating with the pump the waste fluid through the fluid path

Clause 60—The method of any one of clauses 56-58, further including diluting the blood concentration of the waste fluid below a predetermined blood concentration by prefilling the waste container with the waste fluid or another fluid.

Clause 61—A method of arranging a system for collecting waste fluid and for determining a volume of blood collected within the waste fluid, wherein the system including a waste container, a sensor module including a light emitter and an optical detector, a pump in fluid communication with the waste container, a control panel, and a controller in communication with the pump, the sensor module, and the control panel, the method including: positioning a passageway of a cartridge to complete a fluid path with the pump and the waste container with a detection window of the cartridge positioned between the light emitter and the optical detector; and providing an input to the control panel to operate the pump to circulate the waste fluid through the fluid path to be recollected within the waste container, wherein the sensor module is configured to detect an optical characteristic indicative of a blood concentration within the waste fluid.

Clause 62—A method of arranging a system for collecting waste fluid and for determining a volume of blood collected within the waste fluid, wherein the system including a waste container, a manifold receiver, a sensor module including a light emitter and an optical detector, a vacuum source in fluid communication with the waste container, a control panel, and a controller in communication with the vacuum source, the sensor module, and the control panel, the method including: coupling a manifold with the manifold receiver to establish a first fluid path between the manifold and the waste container, wherein the manifold includes a detection window; coupling the sensor module to the manifold such that the light emitter and the optical detector are positioned adjacent or opposite the detection window; and providing an input to the control panel to operate the vacuum source to draw the waste fluid through the manifold and circulate the waste fluid through the first fluid path and a second fluid path is configured to recirculate the waste fluid from the waste container through the manifold to be recollected within the waste container, wherein the sensor module is configured to detect an optical characteristic indicative of a blood concentration within the waste fluid.

Clause 63—The method of clause 62, further including establishing the second fluid path between the manifold and the waste container.

Clause 64—The method of clause 63, wherein the step of establishing the second fluid path further includes coupling a tube to an outlet port of the waste container with an inlet fitting of the manifold.

Clause 65—The method of clause 64, further including coupling a suction tube to another inlet fitting of the manifold.

Clause 66—A method of arranging a medical waste collection system for collecting waste fluid and for determining a volume of blood collected within the waste fluid, wherein the system including a waste container, a manifold receiver, a sensor module including a light emitter and an optical detector, a vacuum source in fluid communication with the waste container, a pump in fluid communication with the waste container, a control panel, and a controller in communication with the vacuum source, the sensor module, and the control panel, the method including: coupling a manifold with the manifold receiver to establish a first fluid path between the manifold and the waste container; positioning a passageway of a cartridge to complete a second fluid path with the waste container with a detection window of the cartridge positioned between the light emitter and the optical detector of the sensor module; and providing an input to the control panel to operate the vacuum source to draw the waste fluid through the manifold and circulate the waste fluid through the first fluid path, and to operate the pump to move the waste fluid through the second fluid path to recirculate the waste fluid from the waste container through the cartridge to be recollected within the waste container, wherein the sensor module is configured to detect an optical characteristic indicative of a blood concentration of the waste fluid.

Clause 67—A cartridge for a medical waste collection system including a waste container defining a waste volume for collecting waste fluid, and a cartridge receiver coupled to the waste container, the cartridge including: a cartridge housing defining a fluid reservoir configured to be arranged in fluid communication with the waste volume with the cartridge removably coupled to the cartridge receiver, and further defining a detection window that is optically clear and in fluid communication with the fluid reservoir; and an actuator coupled to the cartridge housing and configured to be actuated to draw the waste fluid from waste container to within the fluid reservoir; and a sensor module coupled to the cartridge housing to be positioned relative to the detection window so as to detect an optical characteristic indicative of a blood concentration of the waste fluid within the fluid reservoir through the detection window with a light emitter and an optical detector.

Clause 68—The cartridge of clause 67, wherein the actuator is one of a manual actuator configured receive an input from a user, and an electronic actuator configured to be operably controlled by the system.

Clause 69—The cartridge of clause 67 or 68, wherein the actuator is one of a squeeze bulb, a manual syringe, a spring-loaded syringe, a motorized syringe, a pneumatic cylinder, a vacuum source, and a peristaltic pump.

Clause 70—An assembly for detecting an optical characteristic of waste fluid indicative of a blood concentration of the waste fluid with a medical waste collection system including a cartridge receiver, the assembly comprising: a cartridge comprising a cartridge housing comprising a spine defining a detection window, wherein the cartridge is configured to be removably coupled with the cartridge receiver of the medical waste collection system; a sensor housing configured to be removably coupled to the spine; and a sensor module coupled to the sensor housing and comprising emitters and sensors configured to be positioned opposite the detection window.

Clause 71—The assembly of clause 70, wherein the sensor housing includes opposing inner sides formed from a resilient material to be friction fit with the spine of the cartridge.

Clause 72—The assembly of clause 71, wherein the emitters and sensors are coupled to the opposing inner sides of the sensor housing.

Clause 73—The assembly of any one of clauses 70-72, wherein the sensor housing further includes struts coupling the opposing inner sides to define a slot sized to receive the cartridge, and further defining a sensor window for visualization the detection window without removal of the sensor housing.

Clause 74—The assembly of clause 73, further comprising braces configured to secure the cartridge housing and the sensor housing to the medical waste collection system.

Clause 75—The assembly of any one of clauses 70-74, wherein the sensor housing is coupled to a dongle configured to be coupled to a power and data port of the medical waste collection system.

Clause 76—A cartridge configured to be removably coupled with a cartridge receiver of a medical waste collection system for collecting waste fluid under suction, the cartridge comprising: a cartridge housing defining a passageway configured to form a fluid path with the cartridge receiver with the cartridge housing removably positioned within the cartridge opening, wherein the cartridge housing further defines a detection window that is optically clear and in fluid communication with the passageway; and sensor module comprising at least one of a light emitter and an optical detector coupled to the cartridge housing, wherein the light emitter and/or the optical detector is arranged relative to the detection window to provide for detecting an optical characteristic indicative of a blood concentration of the waste fluid being directed through the cartridge.

Clause 77—The cartridge of clause 76, wherein the light emitter and the optical detector are coupled to the cartridge housing.

Clause 78—The cartridge of clause 77, further comprising a controller coupled to the cartridge housing and in electronic communication with the light emitter and the optical detector.

Clause 79—The cartridge of clause 78, further comprising terminals coupled to the cartridge housing and configured to be arranged in electrical contact with complementary terminals of the cartridge receiver, wherein power supplied from the system across the terminals is configured to power at least one of the light emitter, the optical detector, and the controller.

The foregoing description is not intended to be exhaustive or limit the invention to any particular form. The terminology which has been used is intended to be in the nature of words of description rather than of limitation. Many modifications and variations are possible in light of the above teachings and the invention may be practiced otherwise than as specifically described.

1. A cartridge configured to be removably coupled with a cartridge receiver of a medical waste collection system including a sensor housing, and a sensor module for detecting an optical characteristic of waste fluid indicative of a blood concentration of the waste fluid, the cartridge comprising:
a cartridge housing defining a passageway and comprising a head, shoulders extending from the head, and a spine extending from the head and comprising opposing sides that are optically clear and defining a detection window, wherein the spine is configured to be positioned between emitters and sensors of the sensor module with the cartridge housing removably coupled with the cartridge receiver, and wherein a first of the shoulders defines an inlet bore configured to receive the waste fluid being circulated from the medical waste collection system, and a second of the shoulders defines an outlet bore configured to direct the waste fluid being returned to the medical waste collection system after passing through the detection window of the spine.

2. The cartridge of claim 1, wherein the shoulders extend from the head in a first direction, and the spine extends from the head in a second direction opposite the first direction.

3. The cartridge of claim 1, further comprising a gasket coupled to each of the shoulders.

4. The cartridge of claim 1, wherein the head is circular, and wherein the cartridge further comprises an outer gasket coupled to an outer diameter of the cartridge housing.

5. The cartridge of claim 4, wherein axes of the inlet and outlet bores are parallel.

6. The cartridge of claim 2, wherein the cartridge housing further comprises an orientation feature extending from the head in the first direction.

7. The cartridge of claim 1, wherein the cartridge housing is a U-shaped tube.

8. A cartridge configured to be removably coupled with a cartridge receiver of a medical waste collection system including a sensor module for detecting an optical characteristic indicative of a blood concentration of waste fluid, and the cartridge receiver defining a cartridge opening, the cartridge comprising:
a cartridge housing defining an inlet bore, an outlet bore, and a passageway configured to form a circulation fluid path with the cartridge receiver with the cartridge housing removably positioned within the cartridge opening, wherein the cartridge housing further defines a detection window that is optically clear and in fluid communication with the passageway, wherein the detection window is configured to be positioned between the sensor module with the cartridge housing removably positioned within the cartridge opening; and
a retention feature disposed on the cartridge housing and configured to releasably secure the cartridge within the cartridge opening.

9. The cartridge of claim 8, wherein the cartridge housing comprises a head, and shoulders extending from the head and defining bores configured to arranged in fluid communication with conduits of the cartridge receiver to form the circulation fluid path.

10. The cartridge of claim 9, further comprising a tube coupled to the cartridge housing with the tube defining the passageway and the detection window.

11. The cartridge of claim 10, wherein the tube comprises opposing ends coupled to a respective one of the shoulders.

12. The cartridge of claim 9, further comprising a gasket coupled to each of the shoulders.

13. The cartridge of claim 9, further comprising a spine extending from the head in direction opposite the shoulders, wherein the spine defines the detection window.

14. A cartridge for performing quantitative blood loss analysis of waste fluid with a medical waste collection system including a waste container, a vacuum source, a manifold receiver defining a manifold opening, a cartridge receiver defining a cartridge opening, an inlet aperture, and an outlet aperture, the cartridge comprising:
a cartridge housing sized to be removably coupled with the cartridge receiver through the cartridge opening and sized to be unable to be coupled with the manifold receiver through the manifold opening, the cartridge housing comprising a head, and a spine extending from the head to define a detection window, wherein the cartridge housing defines an inlet bore configured to be arranged in fluid communication with the outlet aperture of the cartridge receiver, and an outlet bore configured to be arranged in fluid communication with the inlet aperture of the cartridge receiver, wherein the inlet bore and the outlet bore are on axes parallel to one another.

15. The cartridge of claim 14, wherein the cartridge housing further comprises a first shoulder defining the inlet bore, and a second shoulder defining the outlet bore, wherein the first shoulder and the second shoulder are configured to be coupled to a respective one of the inlet aperture and the outlet aperture of the cartridge receiver.

16. The cartridge of claim 1, further comprising a radiofrequency identification (RFID) tag comprising memory storing data indicative of compatibility of the cartridge with the medical waste collection system.

17-41. (canceled)

42. The cartridge of claim 8, further comprising a radiofrequency identification (RFID) tag comprising memory storing data indicative of compatibility of the cartridge with the medical waste collection system.

43. The cartridge of claim 14, further comprising a radiofrequency identification (RFID) tag comprising memory storing data indicative of compatibility of the cartridge with the medical waste collection system.
